(19) United States
(12) Patent Application Publication
(10) Pub. No.: US 2025/0164358 A1
(43) Pub. Date: May 22, 2025

(54) AFFINITY CAPTURE OF EXTRACELLULAR MATRIX BODIES

(71) Applicant: Aufbau Medical Innovations Ltd., Dublin (IE)

(72) Inventors: John T. G. PENA, New York, NY (US); John IRWIN, North Brunswick Township, NJ (US); Farideh MEHRAEIN-GHOMI, Highland Park, NJ (US); James Murray MITCHELL, New York, NY (US); Harmon Lawrence REMMEL, New York, NY (US)

(21) Appl. No.: 18/929,353

(22) Filed: Oct. 28, 2024

Related U.S. Application Data
(63) Continuation of application No. PCT/US2023/020289, filed on Apr. 27, 2023.
(60) Provisional application No. 63/335,898, filed on Apr. 28, 2022.

(51) Int. Cl. G01N 1/30; G01N 33/68

(52) U.S. Cl. CPC G01N 1/30; G01N 33/6887; G01N 2333/78

(57) ABSTRACT

This invention relates to methods for separating, isolating and/or enriching extracellular matrix bodies in a biological fluid. More particularly, this invention discloses methods for isolating and detecting extracellular matrix bodies from a biological sample as medical information and/or for use in diagnosis and prognosis of disease. The methods include immuno-affinity capture of extracellular matrix bodies.

### CROSS-REFERENCE TO RELATED APPLICATIONS

This application is a continuation of International Patent Application No. PCT/US2023/020289, filed Apr. 27, 2023, which claims priority to U.S. Provisional Patent Application No. 63/335,898, filed Apr. 28, 2022, the entire contents of which are incorporated herein by reference.

### TECHNICAL FIELD

This invention relates to methods for isolating, separating and/or enriching extracellular matrix bodies from biological fluids. More particularly, this invention discloses methods for isolating, detecting and measuring extracellular matrix bodies from a biological sample as diagnostic information and for other uses. The methods include immunoprecipitation of extracellular matrix bodies.

### BACKGROUND

Conventional methods for acquiring diagnostic information from biological samples are limited by the ability to detect features associated with disease. To detect the disease, the source of the biological sample is chosen to be closely connected to disease pathology. For example, intact tissue samples studied in relation to cancer can be excised from a tumor which ensures that the sample is correlated to the disease. Drawbacks of such conventional methods include the need for invasive biopsy for sampling the disease pathology.

Improvements in conventional methods for obtaining diagnostic information from biological samples include studying cells, exosomes or other isolated structures. However, these methods have major limitations when those well-known structures do not readily reflect the disease pathology. This information can have more tenuous connection to the disease and require significant assumptions underlying any diagnostic analysis.

Moreover, conventional methods for separation and analysis of biological fluids do not in general detect all components of the fluid. Most of the fluid and its components are lost, discarded or ignored in conventional methods or assays for nucleic acids or proteins, so that precious material is unused and valuable information is lost.

In addition, specific components of biological fluids are often hidden or masked amongst the many other components, so that any information they reflect is completely missed by conventional methods.

Further, additional components of biological fluids have generally not been exploited in medicine and pharmaceutics. Conventional methods have failed to perceive, appreciate, or examine significant components of biological fluids, in part because their presence is overwhelmed by other features, such as cells, cell components, and/or cell debris. Consequently, conventional methods intentionally or inadvertently discard components obscured by cells in biological fluids. However, such additional significant components of biological fluids can provide a wealth of medical information.

Additional drawbacks of conventional methods include the use of biomarkers which are inherently limited by remote association with disease pathology and significant uncertainties of accurate measurement.

What is needed are methods and compositions for separating, isolating, and/or enriching components obscured in biological fluids for use as new diagnostic information. Methods for isolating and measuring components obscured in biological samples are needed for improving and increasing accuracy of diagnostic analysis relevant to a particular biology and/or disease.

There is an urgent need for methods and compositions that reduce the need for invasive biopsy, which can provide fractions of a biological material having strong connection to disease pathology. Methods for preparing and isolating samples from various biological fluids are needed to expand the range of diagnostic information available toward particular pathologies and disease.

### BRIEF SUMMARY

This invention encompasses methods for separating and isolating biological samples to obtain extracellular components of a biological fluid which are novel features reflecting diagnostic information.

The novel biological components can be associated with a disease and/or closely connected to a disease pathology. More particularly, isolated biological components from bodily fluids can contain substances which inform of a disease, or a lack thereof, and advantageously reduce the need for invasive biopsy for sampling the disease pathology.

Disclosures of this invention include methods for preparing samples for obtaining diagnostic information from biological samples. The methods include separating, enriching and/or isolating structures derived ultimately from an extracellular matrix region. The structures can directly reflect components of disease pathology extant in the isolates. Isolated structures may provide biomarker information with a direct connection to the disease and being useful in diagnostic evaluation and analysis. Methods disclosed herein include biomarker information with significantly enhanced level of detection and/or measurement.

Embodiments of this invention include methods and compositions for separating, isolating, and/or enriching extracellular matrix bodies from biological fluids for diagnostic and/or therapeutic use. The use of extracellular matrix bodies isolated and/or enriched in a biological sample can surprisingly improve diagnostic analysis for a particular biological condition or disease.

Methods and compositions of this disclosure can advantageously reduce the need for invasive patient biopsy by using extracellular matrix bodies which are isolated from biological fluid samples.

This invention includes methods for preparing samples from various biological fluids by and isolating specific components, which surprisingly expands the range of diagnostic information available toward particular pathologies and disease. The fractions obtained from a biological material can have strong connection to disease pathology, or inform of a lack of disease.

This invention can separate, isolate or enrich hidden components including extracellular matrix bodies from biological fluids and unlock their potential for medical information.

Embodiments of this disclosure contemplate methods for preparing samples for a liquid biopsy for diagnosis or prognosis of disease in a subject by isolating extracellular matrix bodies from a biological sample. In some embodiments, the extracellular matrix bodies can be directly associated with the disease.

Additional embodiments of this invention can isolate, extract, and/or utilize extracellular matrix bodies that are a source of multiple and specific biomarkers.

In certain aspects, extracellular matrix bodies can operate as biomarkers through their morphological features. In further aspects, extracellular matrix bodies can operate by containing isolated biochemical markers that may be presented in a disease pathway.

Embodiments of this invention include the following:

Processes for separating, isolating or enriching extracellular matrix bodies in a biological fluid, the process comprising capturing the extracellular matrix bodies on a substrate. The extracellular matrix bodies preferably comprise one or more proteins, and more preferably comprise fibronectin. The substrate may be a solid and comprises one or more immobilized capture moieties for binding and immobilizing the extracellular matrix bodies. The capturing may comprise contacting the biological fluid with the substrate. The capturing may comprise adding the capture moieties to the biological fluid and contacting the biological fluid with the substrate. The biological fluid can be incubated with the substrate, and wherein the substrate comprises capture moieties. The substrate may be of any shape in the form of a bead, a gel, a magnetic bead, a paramagnetic bead, a plate, a well, a membrane, a particle, a sheet, or a fiber. The substrate may preferably be in the form of a magnetic bead or a paramagnetic bead, and more preferably in the form of a magnetic bead. The substrate can be composed of an inorganic material, a polymeric material, an organic material, a metal, a glass, or a combination thereof. The biological fluid may be processed before the capturing on the substrate to remove cells and cell debris.

A “capture moiety” in the context of this invention can be a moiety that has an affinity for one or more components of the extracellular matrix bodies. Some examples of capture moieties include antibodies, metal ions, and dyes. Preferred examples of capture moieties include antibodies which bind specifically to a protein in Table 1 below. The process above may further comprise washing the substrate to remove the biological fluid and any non-bound components from the substrate. The process above may further comprise eluting the immobilized extracellular matrix bodies from the substrate, wherein the extracellular matrix bodies have a principal size from about 1 micrometer to 200 micrometers, or from about 4 micrometers to 200 micrometers. The process above may further comprise tagging the extracellular matrix bodies in the biological fluid for capturing on the substrate. In this context, “tagging” can refer to attaching one or more chemical or biological groups to one or more compounds in the extracellular matrix body to assist with separating, isolating or enriching the extracellular matrix bodies.

The tagging can use epitope tags, affinity tags, fluorescent tags, or a combination thereof.

The process above, wherein the capture moieties bind to any of a protein, an extracellular matrix protein, a polypeptide, a lipid molecule, a lipoparticle, a carbohydrate, or a nucleic acid of the extracellular matrix bodies.

The process above, wherein the capture moieties have specific or non-specific interactions with a component of the extracellular matrix bodies.

The process above, wherein the capture moieties are antibodies, metal ions, or dyes. Preferably, the capture moieties can be antibodies. Thus, the process may preferably use immunoaffinity capture to separate, isolate or enrich the extracellular matrix bodies. When an antibody is used as the capture moiety, the antibody may preferably be a fibronectin antibody.

The process above, wherein at least a majority of the extracellular matrix bodies are captured from the biological fluid, in some variations with an absence of cells.

The process above, wherein substantially all of the extracellular matrix bodies are captured from the biological fluid, in some variations with an absence of cells.

The process above, wherein the captured extracellular matrix bodies are a biomarker or contain biomarkers for medical, diagnostic or prognostic information.

The process above, wherein at least a component of the extracellular matrix bodies is captured from the biological fluid.

The process above, wherein at least a protein component of the extracellular matrix bodies is captured from the biological fluid.

The process above, further comprising additional separating, isolating or enriching of the eluted extracellular matrix bodies by microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

The process above, wherein the extracellular matrix bodies are captured using capture moieties with affinity to one or more protein components of the biological fluid selected from Table 1.

The process above, further comprising adding a reagent to the biological fluid, wherein the reagent is for precipitating the extracellular matrix bodies.

The process above, wherein the extracellular matrix bodies when eluted and re-suspended are at least 5-fold, or at least 10-fold, or at least 100-fold enriched in concentration as compared to the biological fluid.

The process above, wherein the extracellular matrix bodies are associated with a pathology or disease.

The process above, wherein the biological fluid is any one of whole blood, blood plasma, blood serum, cerebrospinal fluid, urine, saliva, sweat, tears, synovial fluid, pleural fluid, gastric fluid, peritoneal fluid, breast milk, nipple aspirate, semen, amniotic fluid, vitreous, aqueous humor, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof. The biological fluid is preferably any one of blood plasma, cerebrospinal fluid, vitreous humor or aqueous humor, and is more preferably blood plasma or cerebrospinal fluid.

The process above, further comprising determining a level of a biomarker of the separated, isolated or enriched extracellular matrix bodies. The biomarker can be the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid. For example, the biomarker can be the quantity of one or more of, for example 1, 2, 3 or 4 of, the proteins set out in Table 1 below. Preferably, the biomarker can be the quantity of fibronectin. More preferably, the biomarker may be the quantity of captured extracellular matrix bodies.

The process above, further comprising determining a level of a biomarker of the separated, isolated or enriched extracellular matrix bodies. The biomarker can be the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid. For example, the biomarker can be the quantity of one or more of, for example 1, 2, 3 or 4 of, the proteins set out in Table 1 below. Preferably, the biomarker is the quantity of fibronectin. More preferably, the biomarker is the quantity of extracellular matrix bodies.

The process above, wherein the level of the substance is determined by any of microscopy, immunostaining, fluorescence assay, chelate complexation, quantitative HPLC, spectrophotometry, antibody array, Western blot, immunoassay, immunoprecipitation, enzyme-linked immunosorbent assay (ELISA), LC-MS, LC-MRM, radioimmunoassay, mass spectrometry, 2D gel mass spectrometry, LC-MS/MS, RT-PCR, nucleic acid sequencing, next generation sequencing, multi-well automated versions thereof, and combinations thereof. The level of the substance is preferably determined by microscopy, immunostaining, or enzyme-linked immunosorbent assay (ELISA).

In some embodiments, the process comprises separating, isolating or enriching extracellular matrix bodies from a biological fluid by capturing extracellular matrix bodies containing fibronectin or a proteomic composition in Table 1 via immunoprecipitation using a magnetic bead kit comprising antibodies to the fibronectin or proteomic composition in Table 1.

In one embodiment, the process comprises separating, isolating or enriching extracellular matrix bodies from blood plasma by capturing extracellular matrix bodies containing fibronectin or a proteomic composition in Table 1 via immunoprecipitation using a magnetic bead kit comprising antibodies to the fibronectin or proteomic composition in Table 1.

In one embodiment, the process comprises separating, isolating or enriching extracellular matrix bodies from cerebrospinal fluid by capturing extracellular matrix bodies containing fibronectin or proteomic composition in Table 1 via immunoprecipitation using a magnetic bead kit comprising antibodies to the fibronectin or a proteomic composition in Table 1.

In one embodiment, the process comprises separating, isolating or enriching extracellular matrix bodies from ocular fluid by capturing extracellular matrix bodies containing fibronectin or proteomic composition in Table 1 via immunoprecipitation using a magnetic bead kit comprising antibodies to the fibronectin or a proteomic composition in Table 1. The ocular fluid may be vitreous humor or aqueous humor.

Embodiments of this invention further contemplate compositions comprising extracellular matrix bodies captured by the process above. The composition above, wherein the extracellular matrix bodies are complexed with a tag or a solid substrate. The composition of claim 32, wherein the extracellular matrix bodies are associated with pathology of a disease. A composition above, for use in therapy of a human or animal body.

Additional embodiments of this invention include methods for preparing a biological sample for a medical, diagnostic or prognostic use, the method comprising isolating extracellular matrix bodies from the biological sample, wherein the extracellular matrix bodies have a principal size from about 1 micrometer to 200 micrometers, or from about 4 micrometers to 200 micrometers. The method above, wherein the biological sample is composed of a bodily fluid. The method above, wherein the bodily fluid is any of whole blood, blood plasma, blood serum, cerebrospinal fluid, vitreous, aqueous humor, breast milk, nipple aspirate, urine, saliva, sweat, tears, synovial fluid, pleural fluid, gastric fluid, peritoneal fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof. The method above, wherein the extracellular matrix bodies are isolated by any of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof. The method above, comprising capturing the extracellular matrix bodies on a solid substrate. The method above, wherein the capturing comprises adding capture moieties to the biological sample and contacting the biological sample with the substrate.

Further embodiments of this invention include processes for diagnosing, prognosing or monitoring a disease in a subject, the process comprising
separating, isolating or enriching extracellular matrix bodies in a biological fluid sample of the subject;
determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid; and
comparing the level of the biomarkers to a reference level based on a control group of subjects, and diagnosing, prognosing or monitoring the disease in the subject. The separating, isolating or enriching may be performed according to the steps of the methods above.

The process above, wherein the separated, isolated or enriched extracellular matrix bodies comprise biomarkers in the form of proteins, extracellular matrix proteins, polypeptides, lipids, lipoparticles, carbohydrates, nucleic acid molecules, DNA, or an expression level of a nucleic acid. The process above, wherein the separating, isolating or enriching extracellular matrix bodies in a biological fluid sample of the subject comprises using capture moieties to immobilize extracellular matrix bodies on a substrate. The process above, comprising treating the subject for the disease by any one or more of surgery, drug therapy, therapeutic radiation, and chemotherapy.

This invention further provides kits for separating, isolating or enriching extracellular matrix bodies in a biological fluid, comprising: a container for holding the biological fluid; and one or more reagents for capturing the extracellular matrix bodies on a solid substrate. The kit above, wherein the reagents have affinity for a protein component of the extracellular matrix bodies. The kit above, wherein the reagents are suitable for any of microscopy, immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid sequencing, next generation sequencing, multi-well automated versions thereof, and combinations thereof.

### BRIEF DESCRIPTION OF THE DRAWINGS

FIG. 1 shows a flow chart of steps of an embodiment for separating, isolating or enriching extracellular matrix bodies from a biological fluid by affinity techniques.

FIG. 2 shows a schematic of an embodiment for separating, isolating or enriching extracellular matrix bodies from a biological fluid by affinity techniques.

FIG. 3 shows a flow chart of steps of an embodiment for separating, isolating or enriching extracellular matrix bodies from a biological fluid by affinity techniques.

FIG. 4 shows a schematic of an embodiment for separating, isolating or enriching extracellular matrix bodies from a biological fluid by affinity techniques.

FIG. 5 shows a fluorescence photomicrograph of extracellular matrix bodies isolated from human blood plasma by immunoprecipitation with a galectin-3-binding protein antibody conjugated to a magnetic bead (10.1 ng/ml of Gal3BP). A control antibody (IgG) showed no isolation of extracellular matrix bodies (not shown).

FIG. 6 shows a graph of the quantities of extracellular matrix bodies isolated from human cerebrospinal fluid by immunoprecipitation with a probe antibody (Ab) conjugated to a magnetic bead. The relative abundance of extracellular matrix bodies (y-axis) was proportional to the probe antibody concentration (pg/ml). A negative control antibody (IgG) showed no isolation of extracellular matrix bodies. These data show that immunoprecipitation of extracellular matrix bodies was far greater using the specific probe antibodies than for the negative control.

FIG. 7 shows a graph of the quantities of extracellular matrix bodies isolated from human blood plasma by immunoprecipitation with a galectin-3-binding protein antibody conjugated to a magnetic bead (18.7 ng/ml of Gal3BP). The relative abundance of extracellular matrix bodies (y-axis) was proportional to the Gal3BP antibody concentration (pg/ml). A negative control antibody (IgG) showed no isolation of extracellular matrix bodies. These data show that immunoprecipitation of extracellular matrix bodies was far greater using the specific probe antibodies than for the negative control.

FIG. 8 shows a histogram of the sizes and relative abundances of extracellular matrix bodies isolated from human blood plasma by immunoprecipitation with a galectin-3-binding protein antibody conjugated to a magnetic bead (18.7 ng/ml of Gal3BP). The graph shows the abundance of extracellular matrix bodies (y-axis) determined by analyzing multiple (n=3) representative fluorescence photomicrographs using computational software (ImageJ). The sizes of extracellular matrix bodies in FIG. 8 ranged from 138 (μm)² (about 13.2 μm diameter) to 681 (μm)² (about 29 μm diameter) and greater, as quantified from photomicrographs.

FIG. 9 shows a graph of the quantities of extracellular matrix bodies isolated from a biological fluid sample, bovine vitreous humor, using immunoprecipitation. Fibronectin is a component of extracellular matrix bodies of the vitreous humor. FIG. 9 shows the relative abundance of isolated extracellular matrix bodies by assay of the amount of fibronectin protein (ng/ml, y-axis) after incubation of the vitreous humor with antibodies specific for fibronectin conjugated to magnetic beads. Also shown is measurement using a comparative negative control IgG antibody, which is essentially zero. These data show that fibronectin immunoprecipitation of extracellular matrix bodies (10.1 ng/ml, black bar) was far greater than for negative control IgG antibody (0.2 ng/ml, white bar).

FIG. 10 shows a representative fluorescence photomicrograph of native bovine vitreous humor. Extracellular matrix bodies were present and visualized with immunofluorescent staining for fibronectin (black regions, anti-fibronectin antibody, Alexa 488). The bovine vitreous humor was fixed to a glass slide, incubated with a fibronectin antibody conjugated to a fluorophore (Alexa 488), and imaged with wide-field fluorescence microscopy (FITC).

FIG. 11 shows a representative fluorescence photomicrograph of extracellular matrix bodies extracted from a biological fluid, bovine vitreous humor, after immunoprecipitation with fibronectin antibody. Antibodies specific to fibronectin and conjugated to magnetic beads were incubated with bovine vitreous humor to extract extracellular matrix bodies. The sample was washed for non-specific binding and visualized on a glass slide for imaging.

FIG. 12 shows a representative fluorescence photomicrograph for corresponding negative control as compared to FIG. 11. FIG. 12 confirms that essentially no extracellular matrix bodies were found after immunoprecipitating with a control IgG antibody.

FIG. 13 shows a graphical representation of the relative amounts of extracellular matrix bodies isolated from bovine vitreous humor using immunoprecipitation with fibronectin or control antibodies conjugated to magnetic beads. The graph shows the relative abundance of extracellular matrix bodies (y-axis) determined by analyzing multiple (n=3) representative fluorescence photomicrographs using computational software (ImageJ). Analysis was done with representative photographic fields for each 20× photomicrographic image. The relative amount of extracellular matrix bodies was measured as a percent of total. FIG. 13 shows that the fibronectin immunoprecipitation sample contained 93.4% of the total of extracellular matrix bodies (black bar) relative to control.

### DETAILED DESCRIPTION OF THE INVENTION

This invention provides methods and compositions for separating, isolating, and/or enriching extracellular matrix bodies from biological fluids. The extracellular matrix bodies can provide diagnostic information from biological samples which include features associated with a disease. The biological features can be closely connected to the disease pathology. More particularly, biological features of substances isolated from samples such as bodily fluids can inform of the disease and advantageously reduce the need for invasive biopsy for sampling the disease pathology.

Embodiments of this invention include methods for distinguishing extracellular matrix bodies in a biological fluid. The method allows for isolating, separating, depleting and/or enriching extracellular matrix bodies from biological fluids. More particularly, this invention discloses methods for isolating, detecting and measuring extracellular matrix bodies from a biological sample as diagnostic information and for other uses. The methods include antibody-based affinity capture of extracellular matrix bodies. The methods can also include capturing extracellular matrix bodies via their components. Because extracellular matrix bodies may contain nucleic acids, the extracellular matrix bodies may be captured by affinity and analysis methods for nuclei acids, such as nucleic acid probes, nucleic acid isolation and/or purification methods, and aptamer and SELEX methodologies.

Disclosures of this invention include methods for obtaining diagnostic information from biological samples by studying structures isolated from components of an extracellular matrix found in a biological fluid. The structures may readily reflect components of disease pathology extant in the isolates. The structures provide information with a direct connection to the disease and are useful in diagnostic analysis.

This invention can further provide methods for obtaining biomarker information having direct association with a disease pathology. Methods disclosed herein include biomarker information with significantly enhanced level of measurement.

Embodiments of this invention include methods and compositions for separating, isolating, and/or enriching extracellular matrix bodies from biological fluids for use as diagnostic information. The use of extracellular matrix bodies isolated and/or enriched in a biological sample can surprisingly increase diagnostic analysis for a particular biological condition or disease.

Methods and compositions of this disclosure can advantageously reduce the need for invasive patient biopsy because extracellular matrix bodies are isolated from bodily fluid samples.

This invention includes methods for preparing and isolating samples from various biological fluids which surprisingly expands the range of diagnostic information available toward particular pathologies and disease. The fractions obtained from a biological material can have strong connection to disease pathology.

Embodiments of this disclosure provide methods for preparing samples for a liquid biopsy for diagnosis or prognosis of disease in a subject, by isolating extracellular matrix bodies from a biological sample from the subject, wherein the extracellular matrix bodies are associated with the disease.

In some aspects, this disclosure shows methods for obtaining extracellular matrix bodies from biological fluids. The extracellular matrix bodies are novel structures having uses in diagnostics and development of new therapeutics, as well as for processing of bodily fluids for medical or commercial use.

In further aspects, this disclosure includes methods for separating, isolating and/or enriching extracellular matrix bodies from biological fluids.

In additional aspects, extracellular matrix bodies of this disclosure can be surprisingly well separated from cells. Extracellular matrix bodies can also be surprisingly well separated from nano-vesicles, which are much smaller.

Methods of this invention can provide a novel window into disease pathology by separating, isolating and/or enriching extracellular matrix bodies for analysis of their properties and structure.

In general, conventional methods for analyzing a bodily fluid and isolating or purifying its components do not identify large extracellular matrix bodies as exemplified in this disclosure. Conventional methods intentionally or inadvertently discard extracellular matrix bodies.

This invention provides methods and compositions for sampling dynamic extracellular matrix structures and/or disease pathology through their presentation in bodily fluids. Extracellular matrix bodies provided by this disclosure reflect the diversity of extracellular matrix structures that determine tissue properties. Such extracellular matrix structures can be highly dynamic and constantly deposited, remodeled, and degraded to maintain tissue homeostasis. The extracellular matrix structures are spatiotemporally regulated to control cell behavior and differentiation, and dysregulation of extracellular matrix structures can lead to disease pathology.

Processes of this disclosure for separating, isolating, and/or enriching extracellular matrix bodies can be useful for identifying biomarkers of disease and therapies thereof, as well as concentrating or purifying biological fluids.

In some embodiments, a process for diagnosing, prognosing or monitoring a disease in a subject, which is performed by separating, isolating or enriching extracellular matrix bodies in a biological fluid, can include treating the subject for the disease by any one or more of surgery, drug therapy, therapeutic radiation, and chemotherapy.

As used herein, the term separating can include depleting and/or removing extracellular matrix bodies from a biological fluid.

Methods of this invention can provide advantageously intact biomarkers from biological fluids.

Methods of this invention can further provide advantageously stable fractions of extracellular matrix bodies and biomarkers therefrom.

### Extracellular Matrix Bodies

This invention discloses methods for isolating extracellular matrix bodies from a biological sample. Extracellular matrix bodies can be associated with a disease in a subject, or with a non-disease subject, and can provide markers for disease. Different bodily fluids can provide biological samples containing extracellular matrix bodies related to a biology of interest.

While not wishing to be bound by theory, the presence of extracellular matrix bodies has generally not been exploited in medicine and pharmaceutics. Conventional methods have failed to perceive, appreciate, or examine extracellular matrix bodies, in part because their presence is overwhelmed by other features, such as cells, cell components, or cell debris. Further, conventional methods intentionally or inadvertently discard extracellular matrix bodies. In the absence of cells, cell components, and/or cell debris, it has been discovered that extracellular matrix bodies can separated, isolated, and/or enriched from a biological fluid to provide a wealth of medical information. Further, the dynamic nature of the heterogeneous structure and properties of extracellular matrix bodies has been a barrier to separating, isolating and/or enriching extracellular matrix bodies for uses in medicinal fields. Methods, compositions and discoveries described herein provide novel approaches to obtaining and utilizing extracellular matrix bodies.

As used herein, the terms extracellular matrix bodies can refer to a morphologically and physiologically distinct heterogeneous mass of substances which may form a bioparticle. Extracellular matrix bodies can have various shapes with principal sizes, length or width, ranging from about 1 micrometer up to hundreds of micrometers.

In certain embodiments, extracellular matrix bodies can have a principal size ranging from about 4 micrometers up to hundreds of micrometers.

An extracellular matrix body bioparticle can be suspended in a biological fluid, from which it can be separated, isolated or enriched. Extracellular matrix bodies may be composed of proteins, extracellular matrix proteins, polypeptides, lipid molecules, lipoparticles, carbohydrates, and combinations thereof. Certain components of an extracellular matrix body may be composed of nucleic acids, including any of the various forms of DNA and/or RNA. Extracellular matrix bodies may contain portions of extracellular matrix tissue structures.

The morphology of extracellular matrix bodies can range from diffuse, wherein the body may be composed of extended arms of various lengths, to a more compacted structure, wherein the body may be composed of closely-packed components; and to a more continuous structure, wherein the body may be composed of a substantially continuous mass.

The morphology of extracellular matrix bodies of a biological fluid can be related to a disease, condition, pathology, or non-disease state of a subject.

The morphology of extracellular matrix bodies can be dynamic and can change with circumstances. The morphology of extracellular matrix bodies may depend on environment, such as the biological fluid in which it is found, as well as processes to which it has been subjected, such as circulation or transport in an organism or laboratory or industrial processes. The shape and/or size of extracellular matrix bodies can vary with the environment, such as fluid temperature, pressure, flow, viscosity, ionicity, pH, osmolality, and composition.

Extracellular matrix bodies can present biomarkers of various kinds which can be useful as diagnostic information. Extracellular matrix bodies themselves can operate as biomarkers through their quantitative and morphological features.

The size of extracellular matrix bodies can be determined by microscopy, hydrodynamic radius, hydrodynamic volume, or radius of gyration, as well as by size fractionation methods and dynamic light scattering. The size and shape can be determined by microscopy methods. Density, mass and charge can be determined by hydrodynamic methods, light scattering methods, particle tracking methods, and electrophoretic measurements.

In some biological fluids, extracellular matrix bodies may include various regularly-shaped microparticles or nanoparticles, typically less than about 1 micrometer in dimension, as well as irregularly shaped substances that can be attached or adhered within a body. The structures of certain components of an extracellular matrix body may include membranes, layers, or bilayers.

In certain bodily fluids, an extracellular matrix body may contain a cell, such as a cell from a component of an extracellular matrix. Examples of a cell include a stromal cell, a fibroblast, an immune cell, a tumor cell, a mesenchymal cell, a vascular cell, and various other cells such as compromised or diseased cells found in bodily fluids.

In some bodily fluids, an extracellular matrix body may include within its heterogeneous structure various components such as microparticles, nanoparticles, vesicles, extracellular vesicles, various small “mere” particles, exosomes, endosomes, organelles, fibers, fibrous structures, and/or secretions of various cells or tissues. However, extracellular matrix bodies are in general larger than such particles and components.

In some aspects, extracellular matrix bodies isolated by the methods herein may be at least about 1 micrometer in size, or at least about 2 micrometers, or at least about 4 micrometers, or at least about 5 micrometers, or at least about 10 micrometers, or at least about 25 micrometers, or at least about 50 micrometers, or at least about 150 micrometers, or at least about 200 micrometers in a principal size.

Among other things, micrograph images of extracellular matrix bodies in a bodily fluid, suspended in solution, re-suspended in buffer, retained by fixation on a slide or substrate, or isolated in a microfluidic device can provide a measure of principal size.

In certain embodiments, extracellular matrix bodies isolated by the methods herein may be about 1 to 50 micrometers in size, or about 1 to 200 micrometers, or about 2 to 200 micrometers, or about 4 micrometers to 200 micrometers, or about 4 to 300 micrometers, or about 5 to 500 micrometers in a principal size.

### Affinity Isolation of Extracellular Matrix Bodies

In some aspects, this disclosure provides methods for separating, isolating and/or enriching extracellular matrix bodies from bodily fluids by affinity separation or affinity chromatography.

Some methods for affinity chromatography are given in S. Reichelt, Affinity Chromatography (2015 Springer); D. Hage et al, Handbook of Affinity Chromatography (2005 CRC Press).

In further aspects, this disclosure provides methods for separating, isolating and/or enriching extracellular matrix bodies from bodily fluids by any one of many known formats for affinity separation and/or affinity chromatography.

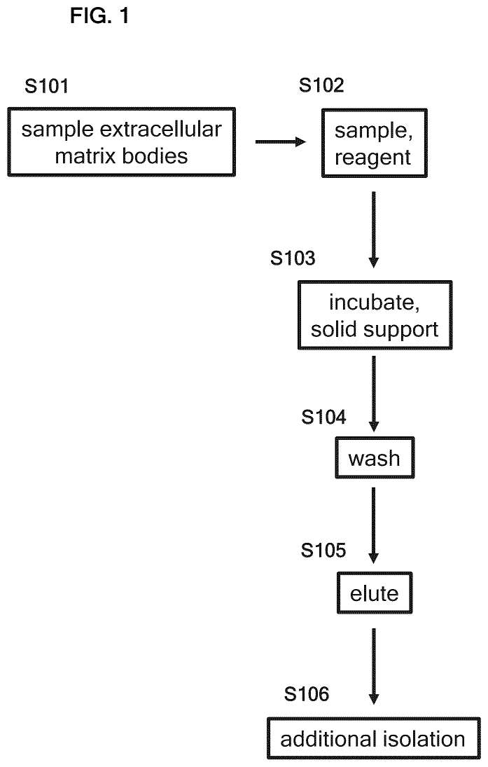

FIG. 1 shows steps of methods to capture extracellular matrix bodies from a sample of bodily fluid. A sample of bodily fluid can be prepared to provide extracellular matrix bodies in step S101. The bodily fluid may contain extracellular matrix bodies when obtained from a subject. Methods of this invention can separate, isolate and/or enrich extracellular matrix bodies from a sample of native bodily fluid.

Alternatively, a sample of bodily fluid may be processed in step S101 for removing cells and cell debris. For example, a sample of a bodily fluid can be processed by any one or more of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof, to remove cells and cell debris. The alternative step of removing cells and cell debris may enhance measurement of extracellular matrix bodies and biomarkers.

In some embodiments, a sample of bodily fluid can be prepared by adding a reagent in step S102. Examples of reagents include a gelling agent, a surfactant, or reagents for interacting with biological components of the sample.

Step S103 includes contacting and/or incubating the sample of bodily fluid with a solid support. Examples of a solid support include beads, gels, magnetic beads, paramagnetic beads, plates, membranes, particles, sheets, and fibers, as used in many formats of chromatography. The solid support can be of any shape and be composed of an inorganic material, a polymeric material, an organic material, a metal, a glass, or a combination thereof. Examples of a polymeric material include agarose, dextran, polyacrylamide, and crosslinked structures thereof.

After contacting and/or incubating the sample of bodily fluid with the solid support, the solid support can be washed in step S104 to remove non-bound molecules from the sample.

Extracellular matrix bodies and components thereof can be eluted from the solid support in step S105.

After elution from the solid support, extracellular matrix bodies and components thereof can be further separated, isolated, enriched, or purified in step S106 by any of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

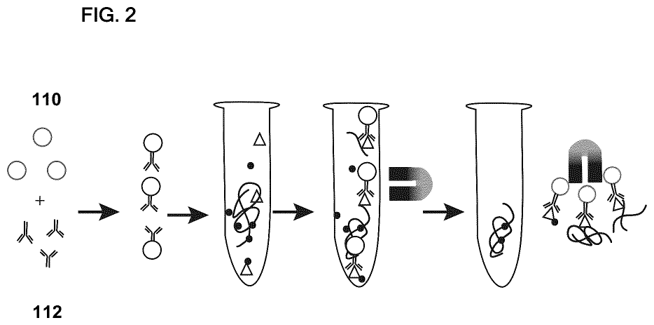

FIG. 2 shows a schematic of an embodiment for separating, isolating or enriching extracellular matrix bodies from a bodily fluid by immunoaffinity capture. Magnetic beads 110 were crosslinked to fibronectin or control IgG antibodies 112 and then added to a vial of homogenized bovine vitreous humor. The magnetic beads with ECM bodies were extracted from the solution using a magnetic stand.

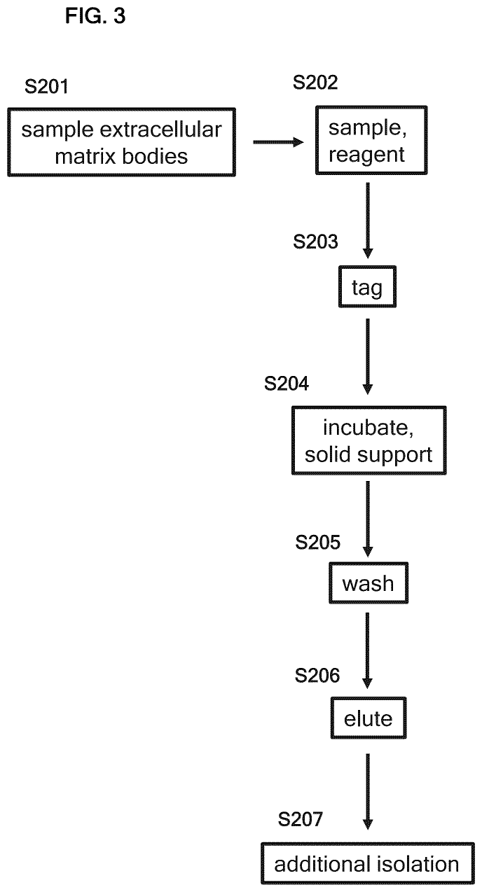

FIG. 3 shows steps of methods to capture extracellular matrix bodies from a sample of bodily fluid. A sample of bodily fluid can be prepared to provide extracellular matrix bodies in step S201. The bodily fluid may contain extracellular matrix bodies when obtained from a subject. Methods of this invention can separate, isolate and/or enrich extracellular matrix bodies from a sample of native bodily fluid.

Alternatively, a sample of bodily fluid may be processed in step S201 for removing cells and cell debris. For example, a sample of a bodily fluid can be processed by any one or more of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof, to remove cells and cell debris. The alternative step of removing cells and cell debris may enhance measurement of extracellular matrix bodies and biomarkers.

In some embodiments, a sample of bodily fluid can be prepared by adding a reagent in step S202. Examples of reagents include a gelling agent, a surfactant, or reagents for interacting with biological components of the sample.

In some embodiments, a sample of bodily fluid can be tagged in step S203. Examples of tags include epitope tags, affinity tags, fluorescent tags, and combinations thereof.

Step S204 includes contacting and/or incubating the sample of bodily fluid with a solid support. Examples of a solid support include beads, gels, magnetic beads, paramagnetic beads, plates, membranes, particles, sheets, and fibers, as used in many formats of chromatography. The solid support can be of any shape and be composed of an inorganic material, a polymeric material, an organic material, a metal, a glass, or a combination thereof.

After contacting and/or incubating the sample of bodily fluid with the solid support, the solid support can be washed in step S205 to remove non-bound molecules from the sample.

Extracellular matrix bodies and components thereof can be eluted from the solid support in step S206.

After elution from the solid support, extracellular matrix bodies and components thereof can be further separated, isolated, enriched, or purified in step S207 by any of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

In some embodiments, extracellular matrix bodies can be obtained by a separation or isolation process from a bodily fluid. Some examples of methods for obtaining samples of extracellular matrix bodies from a bodily fluid include microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

In further embodiments, processing of a bodily fluid can include a step for separating, isolating or enriching extracellular matrix bodies by centrifugation and/or filtration. For example, a biological fluid can be centrifuged to apply less than about 1,200 g forces for at least about three minutes. In certain embodiments, the centrifugation step may be performed at 500 to 5,000 g for less than about ten minutes. Centrifugation steps can be combined with filtration. The processing step can remove cells and other large components that are not attached to extracellular matrix bodies.

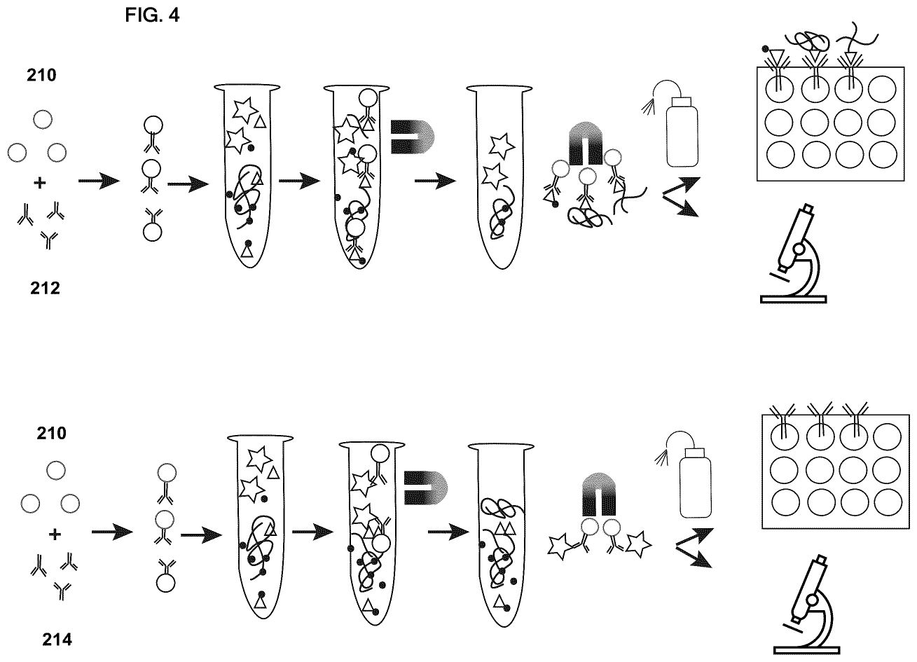

FIG. 4 shows a schematic of an embodiment for separating, isolating or enriching extracellular matrix bodies from a bodily fluid by immunoaffinity capture. Magnetic beads 210 were crosslinked to fibronectin 212 or control IgG 214 antibodies and then added to a vial of homogenized bovine vitreous humor. The magnetic beads attached to immunocomplexes were separated from the solution using a magnetic stand. Fibronectin and IgG bound to the beads were recovered after incubation with an elution buffer. ELISA assays were conducted on the eluates. An aliquot of each eluate was fixed to a glass slide and imaged by microscopy.

### Methods for Isolating Extracellular Matrix Bodies

This disclosure provides methods for separating, isolating and/or enriching extracellular matrix bodies from bodily fluids by affinity techniques.

Methods of this invention for separating, isolating, and/or enriching extracellular matrix bodies can provide surprisingly intact biomarkers from bodily fluids.

Methods of this invention for separating, isolating, and/or enriching extracellular matrix bodies can provide surprisingly stable fractions of extracellular matrix bodies and biomarkers which they present.

In some embodiments, this invention includes processes for separating, isolating or enriching extracellular matrix bodies in a biological fluid by capturing the extracellular matrix bodies on a solid substrate.

Examples of a solid substrate include an inorganic material, a polymeric material, an organic material, a metal, a glass, or a combination thereof. The solid substrate can be of any shape, for example, a bead, a gel, a magnetic bead, a paramagnetic bead, a plate, a membrane, a particle, a sheet, or a fiber, as well as shapes known and used in the field.

In some aspects, the solid substrate may carry immobilized capture moieties for binding and immobilizing the extracellular matrix bodies, which can be done by contacting and/or incubating the biological fluid with the solid substrate. The extracellular matrix bodies may have a principal size from about 1 micrometer to 200 micrometers, or from about 4 micrometers to 200 micrometers.

Capture moieties may have specific or non-specific interactions with a component of the extracellular matrix bodies. Examples of capture moieties include antibodies, metal ions, and dyes. Examples of capture moieties include antibodies which bind specifically to a protein in Table 1 below, or molecules having affinity for such proteins. Capture moieties that are antibodies may be monoclonal or polyclonal. In some embodiments, a capture moiety may bind to more than one, or to a plurality of extracellular matrix bodies via components of the extracellular matrix bodies, such as proteins, extracellular matrix proteins, polypeptides, lipids, lipoparticles, carbohydrates, and nucleic acid molecules. In certain embodiments, a capture moiety may bind to a cell embedded in an extracellular matrix body.

In further embodiments, extracellular matrix bodies in the biological fluid may be tagged for capturing on the substrate. Extracellular matrix bodies may be tagged with any of epitope tags, affinity tags, fluorescent tags, or a combination thereof.

In some embodiments, a reagent may be added to a biological fluid in a step of a method of this invention. Examples of reagents include buffers, lysing solutions, nucleic acid cleavage agents or cleavage inhibitors, precipitation agents, and fixative reagents. In certain embodiments, reagents may include any of a carrier fluid, a biofluid, water, purified water, saline solution, organic solvents, a gelling agent, a surfactant, and combinations thereof. In additional embodiments, reagents may include one or more reagents for measuring a biomarker level or quantity, or for comparing a biomarker level to a control.

In some embodiments, a step of comparing the level of a biomarker to a reference level based on a control group of subjects can include a step of determining differences between a level of a biomarker and a reference level. A difference between a level of a biomarker and a reference level may also be a deviation of a level of a biomarker from a reference level.

Examples of reagents include one or more reagents for measuring one or more proteins disclosed in Table 1 herein. Examples of reagents include reagents for amplifying a nucleic acid. In further embodiments, reagents may include reagents for co-immunoprecipitation. In further embodiments, reagents may include ligands for binding or associating with a component of extracellular matrix bodies of a biological fluid.

In some aspects, methods for separating, isolating and/or enriching extracellular matrix bodies of a biological fluid may include competitive elution in which a competitive ligand is introduced to elute captured extracellular matrix bodies. In certain embodiments, captured extracellular matrix bodies may be eluted or released from capture by changing pH, ionic strength, or polarity of a fluid.

In certain aspects, extracellular matrix bodies may be captured from a biological fluid by adding capture moieties to the biological fluid and contacting and/or incubating the mixture with the solid substrate.

In additional aspects, a biological fluid of interest may contain cells and/or cell debris which can be removed to improve the separation, isolation and/or enrichment of extracellular matrix bodies from the biological fluid. In certain embodiments, a biological fluid can be processed to remove cells and cell debris, after which step extracellular matrix bodies can be isolated. Methods for removing cells include centrifugation and filtration.

Having captured extracellular matrix bodies from a biological fluid with capture moieties, a solid substrate can be washed to remove the biological fluid and any non-bound components from the solid substrate. The bound or immobilized extracellular matrix bodies can be eluted from the solid substrate. The extracellular matrix bodies may have a principal size from about 1 micrometer to 200 micrometers, or from about 4 micrometers to 200 micrometers.

In alternative embodiments, extracellular matrix bodies in the biological fluid can be tagged for capturing on the solid substrate. The tag can be paired for affinity to a capture moiety, which can be immobilized on a solid substrate. Examples of tags include epitope tags, affinity tags, fluorescent tags, and combinations thereof. In some embodiments, a tag can be a tagged-protein as bait for binding extracellular matrix bodies.

In any of the foregoing capture formats and methods, the capture moieties can have affinity to, or bind to any of a protein, an extracellular matrix protein, a polypeptide, a lipid molecule, a lipoparticle, a carbohydrate, or a nucleic acid of the extracellular matrix bodies. The interactions by which the capture moieties have affinity to, or bind to extracellular matrix bodies can be specific or non-specific interactions. Examples of capture moieties involving non-specific interactions include metal ions and dyes. Examples of capture moieties may include binding reporter moieties as are known in the art.

### Isolating Extracellular Matrix Bodies

Extracellular matrix bodies are not found in cells and are not a part of cellular structure. Extracellular matrix bodies are heterogeneous bodies found in bodily fluids. In some examples, the structure of extracellular matrix bodies can be diffuse, or compacted, or a substantially continuous mass. Extracellular matrix bodies may be composed of several components, for example, various extracellular proteins, as well as certain nucleic acid molecules and various fibers or strands. Extracellular matrix bodies vary greatly in size and shape over a wide range. These features of structure can make it difficult or impossible to separate, isolate or enrich extracellular matrix bodies from a biological sample.

Further, the morphology of extracellular matrix bodies can be dynamic and can change with circumstances. Because of the dynamic nature, it is unpredictable whether various methods would be successful in separating, isolating or enriching extracellular matrix bodies from a biological sample.

Extracellular matrix bodies differ substantially from cells in density and range of sizes, shapes and structures. Embodiments of this invention include methods for separating, isolating or enriching extracellular matrix bodies by taking advantage of these differences in structure and properties. For example, cells and cell debris can be separated from extracellular matrix bodies by low speed centrifugation, and in turn, extracellular matrix bodies can be selectively separated from the remainder of a biological fluid sample by affinity methods of this disclosure.

Alternatively, in some embodiments, extracellular matrix bodies can be selectively separated from a biological fluid sample by affinity methods of this disclosure, regardless of the presence of cells and/or cell debris in the biological fluid sample. For example, in some embodiments, extracellular matrix bodies can be selectively separated from a biological fluid sample by specific interactions of a capture moiety with at least a protein component of the extracellular matrix bodies.

Embodiments of this invention provide methods for capturing and isolating at least a majority of the extracellular matrix bodies from a biological fluid. In certain embodiments, the isolate of extracellular matrix bodies can be substantially free of cells.

In further embodiments, methods of this invention can capture and isolate substantially all of the extracellular matrix bodies from a biological fluid. In certain embodiments, the isolate of extracellular matrix bodies can have an absence of cells.

Upon re-suspending an isolate of extracellular matrix bodies, the concentration of re-suspended extracellular matrix bodies can be at least 5-fold, or at least 10-fold, or at least 100-fold enriched in concentration as compared to a biological sample, or a native biological fluid.

Examples of a biological fluid include whole blood, blood plasma, blood serum, cerebrospinal fluid, urine, saliva, sweat, tears, synovial fluid, pleural fluid, gastric fluid, peritoneal fluid, breast milk, nipple aspirate, semen, amniotic fluid, vitreous, aqueous humor, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof.

### Biomarker Information of Extracellular Matrix Bodies

Captured extracellular matrix bodies may be a biomarker, or may contain biomarkers for medical, diagnostic or prognostic information.

In some embodiments, a level reflecting the quantity of mass of the separated, isolated or enriched extracellular matrix bodies can be a biomarker.

In further embodiments, a biomarker can be the level of a substance found in the extracellular matrix bodies. Examples of substances include proteins, polypeptides, lipid molecules, lipoparticles, carbohydrates, nucleic acid molecules, or an expression level of a nucleic acid.

In some aspects, a level of a substance may be determined by one or more of microscopy, immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, and multi-well automated versions thereof. In some embodiments, these techniques may include use of multi-well automated systems, for example, automated use of 24, 48, 96 or greater multi-well microplates.

As used herein, the term wells can refer to wells of any shape, depth, volume or geometry, such as microwells of a multi-well plate or array, any of which may be covered or sealed or exposed.

In further aspects, the level of certain substances, or their nature and/or composition may be determined by nucleic acid analysis or sequencing, or next generation sequencing.

### Medical and Diagnostic Information of Extracellular Matrix Bodies

Methods of this disclosure can provide information for medical, diagnostic, prognostic or disease monitoring purposes through the use of extracellular matrix bodies in a biological fluid.

For example, methods for separating, isolating or enriching extracellular matrix bodies from a sample of a bodily fluid of a subject can provide biomarkers in the form of proteins, extracellular matrix proteins, polypeptides, lipids, lipoparticles, carbohydrates, nucleic acid molecules, DNA, or an expression level of a nucleic acid.

Methods of this disclosure provide for determining a level of one or more biomarkers based on the separated, isolated or enriched extracellular matrix bodies, wherein the biomarker is the level of the extracellular matrix bodies themselves, or the level of a substance found in the extracellular matrix bodies. The levels can be compared to a reference level based on a control.

In some embodiments, a control can be a control group of subjects. In certain embodiments, a control can be an absolute level of a determined component of the extracellular matrix bodies.

In these methods, the comparison may provide a diagnosis, a prognosis or a monitor of a disease in a subject.

In some embodiments, a step of comparing the level of a biomarker to a reference level based on a control group of subjects can include a step of determining differences between a level of a biomarker and a reference level. A difference between a level of a biomarker and a reference level may also be a deviation of a level of a biomarker from a reference level.

Methods of this disclosure include preparing a biological sample for a medical, diagnostic, prognostic, or therapeutic use by isolating extracellular matrix bodies from a biological fluid. The extracellular matrix bodies may be isolated by affinity methods of this disclosure.

This disclosure further provides kits for separating, isolating or enriching extracellular matrix bodies in a biological fluid, including a support substrate for holding the biological fluid and one or more reagents for capturing the extracellular matrix bodies on a solid substrate. The reagents may have affinity for a protein component of the extracellular matrix bodies.

### Compositions

Embodiments of this invention further contemplate compositions of isolates and/or extracellular matrix bodies captured by the processes of this disclosure.

In some embodiments, the compositions may be complexes of extracellular matrix bodies, or components thereof, with a tag or a solid support.

The compositions can be associated with a pathology of a disease. The compositions may further be useful for therapy of the human or animal body.

### Extracellular Matrix Bodies Presenting Biomarkers

This invention can further provide methods for obtaining biomarker information having direct association with a disease pathology. Methods disclosed herein include biomarker information with significantly enhanced level of measurement.

Extracellular matrix bodies present biomarkers of various kinds which can be useful as diagnostic information. Extracellular matrix bodies themselves can operate as biomarkers through their quantitative and morphological features.

A process of this disclosure for separating, isolating or enriching extracellular matrix bodies from a biological fluid can advantageously provide biomarker information for medical, diagnostic or prognostic use. Biomarker information can include the quantity of extracellular matrix bodies obtained from a biological fluid. Biomarker information can include the form or identity of a protein, a polypeptide, a lipid molecule, a lipoparticle, a carbohydrate, a nucleic acid molecule, or an expression level of a nucleic acid associated with extracellular matrix bodies. Biomarker information can include the form or identity of extracellular proteins or nucleic acids associated with extracellular matrix bodies.

Some examples of biomarkers found in extracellular matrix bodies of human plasma include proteins given in Table 1.

TABLE 1
Biomarkers found in extracellular matrix bodies

| No. | GENE | IDENTIFIER |
|---|---|---|
| 1. | IGHG4 | tr\|A0A286YFJ8\|A0A286YFJ8_HUMAN |
| 2. | PIGR | sp\|P01833\|PIGR_HUMAN |
| 3. | IGHA1 | sp\|P01876\|IGHA1_HUMAN |
| 4. | IGLV4-60 | sp\|A0A075B6I1\|LV460_HUMAN |
| 5. | IGHD | sp\|P01880\|IGHD_HUMAN |
| 6. | HBB | sp\|P68871\|HBB_HUMAN |
| 7. | HPR | sp\|P00739\|HPTR_HUMAN |
| 8. | IGKV1-6 | sp\|A0A0C4DH72\|KV106_HUMAN |
| 9. | IGHM | sp\|P01871\|IGHM_HUMAN |
| 10. | HBA1 | sp\|P69905\|HBA_HUMAN |
| 11. | CFB | tr\|A0A0G2JH38\|A0A0G2JH38_HUMAN |
| 12. | LPA | sp\|P08519\|APOA_HUMAN |
| 13. | F12 | sp\|P00748\|FA12_HUMAN |
| 14. | B4E1Z4 | tr\|B4E1Z4\|B4E1Z4_HUMAN (C3/C5 convertase) |
| 15. | IGHV3-33 | sp\|P01772\|HV333_HUMAN |
| 16. | SERPINF2 | sp\|P08697\|A2AP_HUMAN |
| 17. | IGHV2-70D | sp\|A0A0C4DH43\|HV70D_HUMAN |
| 18. | JCHAIN | sp\|P01591\|IGJ_HUMAN |
| 19. | IGHV6-1 | sp\|A0A0B4J1U7\|HV601_HUMAN |
| 20. | TLN1 | sp\|Q9Y490\|TLN1_HUMAN |
| 21. | FGA | sp\|P02671\|FIBA_HUMAN |
| 22. | THBS1 | sp\|P07996\|TSP1_HUMAN |
| 23. | IGHA2 | tr\|A0A286YEY5\|A0A286YEY5_HUMAN |
| 24. | CFH | tr\|A0A0D9SG88\|A0A0D9SG88_HUMAN |
| 25. | IGHV5-51 | sp\|A0A0C4DH38\|HV551_HUMAN |
| 26. | IGHV4-39 | sp\|P01824\|HV439_HUMAN |
| 27. | IGKV3-20 | sp\|P01619\|KV320_HUMAN |
| 28. | IGHV3-53 | sp\|P01767\|HV353_HUMAN |
| 29. | ITIH1 | sp\|P19827\|ITIH1_HUMAN |
| 30. | A2M | sp\|P01023\|A2MG_HUMAN |
| 31. | CFD | tr\|K7ERG9\|K7ERG9_HUMAN |
| 32. | CRP | sp\|P02741\|CRP_HUMAN |
| 33. | CD5L | sp\|O43866\|CD5L_HUMAN |
| 34. | LGALS3BP | sp\|Q08380\|LG3BP_HUMAN |
| 35. | IGKV3-15 | sp\|P01624\|KV315_HUMAN |
| 36. | PON1 | sp\|P27169\|PON1_HUMAN |
| 37. | ORM1 | sp\|P02763\|A1AG1_HUMAN |
| 38. | SLC4A1 | sp\|P02730\|B3AT_HUMAN |
| 39. | APOC4-APOC2 | tr\|K7ER74\|K7ER74_HUMAN |
| 40. | IGKV2-29 | sp\|A2NJV5\|KV229_HUMAN |
| 41. | FGB | sp\|P02675\|FIBB_HUMAN |
| 42. | IGLC2 | sp\|PODOY2\|IGLC2_HUMAN |
| 43. | IGHV3OR15-7 | tr\|A0A075B7D8\|A0A075B7D8_HUMAN |
| 44. | IGHA2 | tr\|A0A0G2JMB2\|A0A0G2JMB2_HUMAN |
| 45. | IGLV8-61 | sp\|A0A075B610\|LV861_HUMAN |
| 46. | IGHV3-21 | sp\|A0A0B4J1V1\|HV321_HUMAN |
| 47. | IGLV4-69 | sp\|A0A075B6H9\|LV469_HUMAN |
| 48. | IGLV1-44 | sp\|P01699\|LV144_HUMAN |
| 49. | C4B | sp\|POCOL5\|CO4B_HUMAN |
| 50. | C7 | sp\|P10643\|CO7_HUMAN |
| 51. | ACTB | sp\|P60709\|ACTB_HUMAN |
| 52. | IGLV3-21 | sp\|P80748\|LV321_HUMAN |
| 53. | PGLYRP2 | sp\|Q96PD5\|PGRP2_HUMAN |
| 54. | IGHV1-18 | sp\|A0A0C4DH31\|HV118_HUMAN |
| 55. | IGKV2D-28 | tr\|A0A5H1ZRS2\|A0A5H1ZRS2_HUMAN (P01615) |
| 56. | IGKV2-24 | sp\|A0A0C4DH68\|KV224_HUMAN |
| 57. | FBLL1 | tr\|A0A0G2JRQ6\|A0A0G2JRQ6_HUMAN (fibrillarin) |
| 58. | IGKC | sp\|P01834\|IGKC_HUMAN |
| 59. | IGHV3-9 | sp\|P01782\|HV309_HUMAN |
| 60. | ITIH4 | sp\|Q14624\|ITIH4_HUMAN |
| 61. | AHSG | sp\|P02765\|FETUA_HUMAN |
| 62. | AGT | sp\|P01019\|ANGT_HUMAN |
| 63. | IGHV1-69 | sp\|P01742\|HV169_HUMAN |
| 64. | IGHV1-3 | sp\|A0A0C4DH29\|HV103_HUMAN |
| 65. | SPTA1 | sp\|P02549\|SPTA1_HUMAN |
| 66. | IGLV2-11 | sp\|P01706\|LV211_HUMAN |
| 67. | IGKV1D-33 | tr\|A0A2Q2TTZ9\|A0A2Q2TTZ9_HUMAN |
| 68. | LRG1 | sp\|P02750\|A2GL_HUMAN |
| 69. | IGHV3-72 | tr\|A0A4W8ZXM2\|A0A4W8ZXM2_HUMAN |
| 70. | FGG | sp\|P02679\|FIBG_HUMAN |
| 71. | APOC1 | tr\|K7ERI9\|K7ERI9_HUMAN |
| 72. | LYZ | sp\|P61626\|LYSC_HUMAN |
| 73. | IGKV1-5 | sp\|P01602\|KV105_HUMAN |
| 74. | IGKV1-27 | sp\|A0A075B6S5\|KV127_HUMAN |
| 75. | IGHV1-46 | sp\|P01743\|HV146_HUMAN |
| 76. | C1R | tr\|A0A3B3ISR2\|A0A3B3ISR2_HUMAN |
| 77. | IGKV1-16 | sp\|P04430\|KV116_HUMAN |
| 78. | C4BPB | sp\|P20851-2\|C4BPB_HUMAN |
| 79. | IGFALS | sp\|P35858\|ALS_HUMAN |
| 80. | IGKV4-1 | sp\|P06312\|KV401_HUMAN |
| 81. | PF4 | sp\|P02776\|PLF4_HUMAN |
| 82. | IGHV1OR15-1 | tr\|A0A075B7D0\|A0A075B7DO_HUMAN |
| 83. | IGKV1D-12 | sp\|P01611\|KVD12_HUMAN |
| 84. | IGKV2-30 | sp\|P06310\|KV230_HUMAN |
| 85. | IGHV3-74 | sp\|A0A0B4J1X5\|HV374_HUMAN |
| 86. | SERPING1 | sp\|P05155\|IC1_HUMAN |
| 87. | IGHV3-43D | sp\|PODP04\|HV43D_HUMAN |
| 88. | APOH | sp\|P02749\|APOH_HUMAN |
| 89. | C1S | sp\|P09871\|C1S_HUMAN |
| 90. | PLG | sp\|P00747\|PLMN_HUMAN |
| 91. | IGLV3-9 | sp\|A0A075B6K5\|LV39_HUMAN |
| 92. | IGHV3-49 | sp\|A0A0A0MS15\|HV349_HUMAN |
| 93. | APOL1 | sp\|O14791\|APOL1_HUMAN |
| 94. | SERPINC1 | sp\|P01008\|ANT3_HUMAN |
| 95. | CFP | sp\|P27918\|PROP_HUMAN |
| 96. | AMBP | sp\|P02760\|AMBP_HUMAN |
| 97. | C6 | sp\|P13671\|CO6_HUMAN |
| 98. | IGHV3-38 | sp\|A0A0C4DH36\|HV338_HUMAN |
| 99. | IGKV2D-29 | tr\|A0A5H1ZRS9\|A0A5H1ZRS9_HUMAN |
| 100. | IGHV4-28 | sp\|A0A0C4DH34\|HV428_HUMAN |
| 101. | ADIPOQ | sp\|Q15848\|ADIPO_HUMAN |
| 102. | IGHV3-73 | sp\|A0A0B4J1V6\|HV373_HUMAN |
| 103. | IGHG3 | tr\|A0A286YES1\|A0A286YES1_HUMAN |
| 104. | CPN1 | sp\|P15169\|CBPN_HUMAN |
| 105. | PROS1 | tr\|A0A3B3ISJ1\|A0A3B3ISJ1_HUMAN |
| 106. | IGHG2 | tr\|A0A286YEY4\|A0A286YEY4_HUMAN |
| 107. | IGHV7-4-1 | sp\|A0A0J9YVY3\|HV741_HUMAN |
| 108. | F2 | sp\|P00734\|THRB_HUMAN |
| 109. | IGLV3-10 | sp\|A0A075B6K4\|LV310_HUMAN |
| 110. | IGLV6-57 | sp\|P01721\|LV657_HUMAN |
| 111. | CFH | sp\|P08603\|CFAH_HUMAN |
| 112. | ALB | sp\|P02768\|ALBU_HUMAN |
| 113. | HRG | sp\|P04196\|HRG_HUMAN |
| 114. | FN1 | sp\|P02751-8\|FINC_HUMAN fibronectin |
| 115. | C3 | sp\|P01024\|CO3_HUMAN |
| 116. | C1QB | tr\|D6R934\|D6R934_HUMAN |
| 117. | F5 | tr\|A0A0A0MRJ7\|A0A0A0MRJ7_HUMAN |
| 118. | GC | sp\|P02774\|VTDB_HUMAN |
| 119. | IGLV1-51 | sp\|P01701\|LV151_HUMAN |
| 120. | PLTP | sp\|P55058\|PLTP_HUMAN |
| 121. | EFEMP1 | sp\|Q12805\|FBLN3_HUMAN |
| 122. | SERPINA5 | sp\|P05154\|IPSP_HUMAN |
| 123. | CPN2 | sp\|P22792\|CPN2_HUMAN |
| 124. | IGKV1-13 | sp\|PODP09\|KV113_HUMAN |
| 125. | IGKV3-11 | sp\|P04433\|KV311_HUMAN |
| 126. | IGLV3-25 | sp\|P01717\|LV325_HUMAN |
| 127. | SHBG | tr\|I3L145\|I3L145_HUMAN |
| 128. | FCGBP | tr\|A0A087WXI2\|A0A087WXI2_HUMAN |
| 129. | AZGP1 | sp\|P25311\|ZA2G_HUMAN |
| 130. | ING4 | tr\|E9PNE3\|E9PNE3_HUMAN |
| 131. | APOD | tr\|C9JF17\|C9JF17_HUMAN |
| 132. | HPX | sp\|P02790\|HEMO_HUMAN |
| 133. | CD14 | sp\|P08571\|CD14_HUMAN |
| 134. | APOE | sp\|P02649\|APOE_HUMAN |
| 135. | IGLV3-19 | sp\|P01714\|LV319_HUMAN |
| 136. | C9 | sp\|P02748\|CO9_HUMAN |
| 137. | APOM | sp\|095445\|APOM_HUMAN |
| 138 | IGKV3-7 | sp\|A0A075B6H7\|KV37_HUMAN |
| 139. | IGLL5 | tr\|A0A0B4J231\|A0A0B4J231_HUMAN |
| 140. | ITIH2 | sp\|P19823\|ITIH2_HUMAN |
| 141. | IGKV1-9 | sp\|A0A0C4DH69\|KV109_HUMAN |
| 142. | ECM1 | sp\|Q16610\|ECM1_HUMAN |
| 143. | IGHV3OR16-12 | tr\|A0A075B7B8\|A0A075B7B8_HUMAN |
| 144. | CLEC3B | tr\|E9PHKOJE9PHKO_HUMAN |
| 145. | APOB | sp\|P04114\|APOB_HUMAN |
| 146. | CFHR2 | tr\|A0A3B3IQ51\|A0A3B3IQ51_HUMAN |
| 147. | IG-unk | tr\|A0A0J9YY99\|A0A0J9YY99_HUMAN |
| 148. | IGKV1D-39 | sp\|P04432\|KVD39_HUMAN |
| 149. | TTR | sp\|P02766\|TTHY_HUMAN |
| 150. | FBLN1 | sp\|P23142\|FBLN1_HUMAN |
| 151. | IGHV3-64 | sp\|A0A075B6Q5\|HV364_HUMAN |
| 152. | KLKB1 | tr\|HOYAC1\|HOYAC1_HUMAN |
| 153. | LUM | sp\|P51884\|LUM_HUMAN |
| 154. | VTN | sp\|P04004\|VTNC_HUMAN |
| 155. | C5 | sp\|P01031\|CO5_HUMAN |
| 156. | PZP | sp\|P20742\|PZP_HUMAN |
| 157. | SERPIND1 | sp\|P05546\|HEP2_HUMAN |
| 158. | F13B | sp\|P05160\|F13B_HUMAN |
| 159. | HABP2 | sp\|Q14520\|HABP2_HUMAN |
| 160. | APOA2 | tr\|V9GYM3\|V9GYM3_HUMAN |
| 161. | SERPINA6 | sp\|P08185\|CBG_HUMAN |
| 162. | IGLL1 | sp\|P15814\|IGLL1_HUMAN |
| 163. | APOA4 | sp\|P06727\|APOA4_HUMAN |
| 164. | IGHV3-66 | sp\|A0A0C4DH42\|HV366_HUMAN |
| 165. | IGKV6D-21 | sp\|A0A0A0MT36\|KVD21_HUMAN |
| 166. | IGHV3-13 | sp\|P01766\|HV313_HUMAN |
| 167. | ALB | tr\|A0A0C4DGB6\|A0A0C4DGB6_HUMAN |
| 168. | IGKV1D-37 | sp\|PODSN7\|KVD37_HUMAN |
| 169. | IGKV1-17 | sp\|P01599\|KV117_HUMAN |
| 170. | ITIH3 | sp\|Q06033\|ITIH3_HUMAN |
| 171. | APCS | sp\|P02743\|SAMP_HUMAN |
| 172. | CPB2 | sp\|Q96IY4\|CBPB2_HUMAN |
| 173. | CFI | tr\|E7ETHOJE7ETHO_HUMAN |
| 174. | C1QC | sp\|P02747\|C1QC_HUMAN |
| 175. | SERPINF1 | sp\|P36955\|PEDF_HUMAN |
| 176. | TGFBI | sp\|Q15582\|BGH3_HUMAN |
| 177. | C8G | sp\|P07360\|CO8G_HUMAN |
| 178. | IGHV2-5 | sp\|P01817\|HV205_HUMAN |
| 179. | APOA1 | sp\|P02647\|APOA1_HUMAN |
| 180. | F9 | sp\|P00740\|FA9_HUMAN |
| 181. | CLU | sp\|P10909-6\|CLUS_HUMAN |
| 182. | KNG1 | sp\|P01042-2\|KNG1_HUMAN |
| 183. | IGLV1-40 | sp\|P01703\|LV140_HUMAN |
| 184. | SERPINA1 | sp\|P01009\|A1AT_HUMAN |
| 185. | C8A | sp\|P07357\|CO8A_HUMAN |
| 186. | IGHG1 | tr\|A0A0A0MS08\|A0A0A0MS08_HUMAN |
| 187. | SELENOP | tr\|A0A182DWH7\|A0A182DWH7_HUMAN |
| 188. | HP | sp\|P00738\|HPT_HUMAN |
| 189. | IGHV4-38-2 | sp\|PODP08\|HVD82_HUMAN |
| 190. | SERPINA4 | sp\|P29622\|KAIN_HUMAN |
| 191. | LBP | sp\|P18428\|LBP_HUMAN |
| 192. | ORM2 | sp\|P19652\|A1AG2_HUMAN |
| 193. | FN1 | sp\|P02751-10\|FINC_HUMAN |
| 194. | F10 | sp\|P00742\|FA10_HUMAN |
| 195. | APOC3 | tr\|BOYIW2\|BOYIW2_HUMAN |
| 196. | CFHR1 | sp\|Q03591\|FHR1_HUMAN |
| 197. | ATRN | sp\|075882\|ATRN_HUMAN |
| 198. | B2M | sp\|P61769\|B2MG_HUMAN |
| 199. | VWF | sp\|P04275\|VWF_HUMAN |
| 200. | F13A1 | sp\|P00488\|F13A_HUMAN |
| 201. | AFM | sp\|P43652\|AFAM_HUMAN |
| 202. | GPX3 | tr\|A0A087X1J7\|A0A087X1J7_HUMAN |
| 203. | FCN3 | sp\|075636\|FCN3_HUMAN |
| 204. | RBP4 | tr\|Q5VY30\|Q5VY30_HUMAN |
| 205. | C8B | sp\|P07358\|CO8B_HUMAN |
| 206. | SERPINA3 | sp\|P01011\|AACT_HUMAN |
| 207. | PRG4 | sp\|Q92954\|PRG4_HUMAN |
| 208. | C4A | sp\|POCOL4\|CO4A_HUMAN |
| 209. | SAA2-SAA4 | tr\|A0A096LPE2\|A0A096LPE2_HUMAN |
| 210. | SERPINA7 | sp\|P05543\|THBG_HUMAN |
| 211. | CP | sp\|P00450\|CERU_HUMAN |
| 212. | A1BG | sp\|P04217\|A1BG_HUMAN |
| 213. | C2 | sp\|P06681\|CO2_HUMAN |
| 214. | IGHV1-2 | sp\|P23083\|HV102_HUMAN |
| 215. | HGFAC | tr\|D6RAR4\|D6RAR4_HUMAN |
| 216. | FETUB | sp\|Q9UGM5\|FETUB_HUMAN |
| 217. | GSN | sp\|P06396\|GELS_HUMAN |
| 218. | SERPINA1 | tr\|A0A024R6I7\|A0A024R617_HUMAN |
| 219. | CIQA | sp\|P02745\|C1QA_HUMAN |
| 220 | C4BPA | sp\|P04003\|C4BPA_HUMAN |
| 221. | IGKV3OR2-268 | tr\|A0A0C4DH90\|A0A0C4DH90_HUMAN |
| 222. | HSPG2 | B6EU51 (B6EU51_HUMAN) |
| 223. | FGG | P02679 (FIBG_HUMAN) |
| 224. | FGA | P02671 (FIBA_HUMAN) |
| 225. | FGB | P02675 (FIBB_HUMAN) |
| 226. | APOC1 | P02654 (APOC1_HUMAN) |
| 227. | LYZ | P61626 (LYSC_HUMAN) |
| 228. | ANG | P03950 (ANGI_HUMAN) |
| 229. | IGFBP5 | P24593 (IBP5_HUMAN) |
| 230. | C1QB | P02746 (C1QB_HUMAN) |
| 231. | F5 | P12259 (FA5_HUMAN) |
| 232 | H1-4 | P10412 (H14_HUMAN) |
| 233. | HBA1 | P69905 (HBA_HUMAN) |
| 234. | KRT10 | P13645 (K1C10_HUMAN) |
| 235. | H1-0 | P07305 (H10_HUMAN) |
| 236. | QSOX1 | O00391 (QSOX1_HUMAN) |
| 237. | HABP2 | Q14520 (HABP2_HUMAN) |
| 238. | KRT9 | P35527 (K1C9_HUMAN) |
| 239 | KRT2 | P35908 (K22E_HUMAN) |
| 240. | VTN | P04004 (VTNC_HUMAN) |
| 241. | H4C1 | P62805 (H4_HUMAN) |
| 242. | KRT1 | P04264 (K2C1_HUMAN) |
| 243. | C8B | P07358 (CO8B_HUMAN) |
| 244. | CIQA | P02745 (C1QA_HUMAN) |
| 245. | MTHFD2 | P13995 (MTDC_HUMAN) |
| 246. | CFHR1 | Q03591 (FHR1_HUMAN) |
| 247. | ACTB | P60709 (ACTB_HUMAN) |
| 248. | HRG | P04196 (HRG_HUMAN) |
| 249. | EEF1A1 | P68104 (EF1A1_HUMAN) |
| 250. | H2AC4 | P04908 (H2A1B_HUMAN) |
| 251. | MMP2 | P08253 (MMP2_HUMAN) |
| 252. | TIMP2 | P16035 (TIMP2_HUMAN) |
| 253. | ATP5F1B | P06576 (ATPB_HUMAN) |
| 254. | HGFAC | Q04756 (HGFA_HUMAN) |
| 255. | ENO1 | P06733 (ENOA_HUMAN) |
| 256. | HSPG2 | P98160 (PGBM_HUMAN) |
| 257. | COL18A1 | P39060 (COIA1_HUMAN) |
| 258. | APOE | P02649 (APOE_HUMAN) |
| 259. | CFH | P08603 (CFAH_HUMAN) |
| 260. | RARRES2 | Q99969 (RARR2_HUMAN) |
| 261. | SERPINA5 | P05154 (IPSP_HUMAN) |
| 262. | SAA4 | P35542 (SAA4_HUMAN) |
| 263. | PEBP4 | Q96S96 (PEBP4_HUMAN) |
| 264. | C1QC | P02747 (C1QC_HUMAN) |
| 265. | CHI3L1 | P36222 (CH3L1_HUMAN) |
| 266. | PON1 | P27169 (PON1_HUMAN) |
| 267. | CLU | P10909 (CLUS_HUMAN) |
| 268. | SERPIND1 | P05546 (HEP2_HUMAN) |
| 269. | PCOLCE | Q15113 (PCOC1_HUMAN) |
| 270. | C8A | P07357 (CO8A_HUMAN) |
| 271. | APOM | 095445 (APOM_HUMAN) |
| 272. | APOA1 | P02647 (APOA1_HUMAN) |
| 273. | APOD | P05090 (APOD_HUMAN) |
| 274. | ECM1 | Q16610 (ECM1_HUMAN) |
| 275. | SELENOP | P49908 (SEPP1_HUMAN) |
| 276. | SERPINA4 | P29622 (KAIN_HUMAN) |
| 277. | KNG1 | P01042 (KNG1_HUMAN) |
| 278. | C9 | P02748 (CO9_HUMAN) |
| 279. | PROS1 | P07225 (PROS_HUMAN) |
| 280. | CFHR2 | P36980 (FHR2_HUMAN) |
| 281. | SPON1 | Q9HCB6 (SPON1_HUMAN) |
| 282. | CLSTN1 | 094985 (CSTN1_HUMAN) |
| 283. | APP | P05067 (A4_HUMAN) |
| 284. | GPX3 | P22352 (GPX3_HUMAN) |
| 285. | RNASE1 | P07998 (RNAS1_HUMAN) |
| 286. | IGFBP6 | P24592 (IBP6_HUMAN) |
| 287. | AL645922.1 | B4E1Z4 (B4E1Z4_HUMAN) NM_001710 |
| 288. | ITIH2 | P19823 (ITIH2_HUMAN) |
| 289. | C5 | P01031 (CO5_HUMAN) |
| 290. | GSN | P06396 (GELS_HUMAN) |
| 291. | APOA2 | P02652 (APOA2_HUMAN) |
| 292. | RBP3 | P10745 (RET3_HUMAN) |
| 293. | HP | P00738 (HPT_HUMAN) |
| 294. | C3 | P01024 (CO3_HUMAN) |
| 295. | APOA4 | P06727 (APOA4_HUMAN) |
| 296. | C6 | P13671 (CO6_HUMAN) |
| 297. | APLP2 | Q06481 (APLP2_HUMAN) |
| 298. | TTR | P02766 (TTHY_HUMAN) |
| 299. | PLG | P00747 (PLMN_HUMAN) |

Embodiments of this invention further contemplate processes for determining a level of a biomarker of the separated, isolated or enriched extracellular matrix bodies. The biomarker may be the level of the extracellular matrix bodies, or the level of a substance found in the extracellular matrix bodies. Examples of substances include proteins, polypeptides, lipid molecules, lipoparticles, carbohydrates, nucleic acid molecules, and expression levels of one or more nucleic acids.

In certain embodiments, the level of extracellular matrix bodies may be determined by microscopy.

In additional aspects, the level of a substance may be determined by any analyte technique including immunostaining, fluorescence assay, chelate complexation, quantitative HPLC, spectrophotometry, antibody array, Western blot, immunoassay, immunoprecipitation, co-immunoprecipitation, ELISA, LC-MS, LC-MRM, radioimmunoassay, mass spectrometry, 2D gel mass spectrometry, LC-MS/MS, RT-PCR, and multi-well automated versions thereof.

In further aspects, the level of certain substances, or their nature and/or composition may be determined by nucleic acid analysis or sequencing, or next generation sequencing.

In some embodiments, the level of a substance of a biological fluid may be determined by immunoassay, protein pull down assay, immunoprecipitation or co-immunoprecipitation assay, or columnar affinity chromatography. For example, ELISA can be used in any one of a competitive format, a sandwich format, an antigen down format, a rapid lateral flow format, or a rapid flowing format. These methods can be used for separating, isolating, or enriching extracellular matrix bodies from a biological fluid through interactions of the assay reagents with one or more components of the heterogenous extracellular matrix bodies.

In further aspects, the level of a substance may be determined by imaging techniques including electron microscopy, stereoscopic microscopy, wide-field microscopy, polarizing microscopy, phase contrast microscopy, multiphoton microscopy, differential interference contrast microscopy, fluorescence microscopy, laser scanning confocal microscopy, multiphoton excitation microscopy, ray microscopy, and ultrasonic microscopy.

In some embodiments, the level of a substance may be determined by imaging techniques including positron emission tomography, optical coherence tomography, computerized tomography, or magnetic resonance imaging.

In some embodiments, the level of a substance may be determined by assay techniques including colorimetric assay, chemiluminescence assay, spectrophotometry, immunofluorescence assay, and light scattering.

Examples of methods for analyzing extracellular matrix bodies include microscopy, mass spectrometry, microarray, nucleic acid amplification, hybridization, fluorescence hybridization, immunohistochemistry, nucleic acid analysis or sequencing, next generation sequencing, flow cytometry, chromatography, electrophoresis, and combinations thereof.

### Extracellular Matrix Bodies and Diagnosis of Disease

Embodiments of this invention can provide processes for diagnosing, prognosing or monitoring a disease in a subject. Biomarker levels obtained by separating, isolating, or enriching extracellular matrix bodies can be used for medical or diagnostic uses.

In some aspects, biomarker levels may be obtained from components of extracellular matrix bodies isolated as described herein. Subsequently, a level of one or more biomarkers based on the extracellular matrix bodies that were separated, isolated or enriched can be determined.

In certain embodiments, a biomarker level can be the quantity of extracellular matrix bodies themselves.

In further embodiments, a biomarker level can be the quantity of a substance found in the extracellular matrix bodies, such as a protein, a polypeptide, a lipid molecule, a lipoparticle, a carbohydrate, a nucleic acid molecule, or an expression level of a nucleic acid.

Processes for diagnosing, prognosing or monitoring a disease in a test subject may compare the level of one or more biomarkers from a sample of the test subject to a reference level based on a control group of subjects. The comparison may result in a diagnosis, prognosis or monitor the state or progression of the disease in the subject.

In some embodiments, a control group may be composed of subjects having the same disease as the test subject. In certain embodiments, a control group may be composed of subjects not clinically known to have a disease similar to the test subject. In further embodiments, a control group may be composed of healthy or non-disease subjects.

In further embodiments, biomarker levels determined from separated, isolated or enriched extracellular matrix bodies can be combined with any number of known biomarkers of a particular disease to improve processes for diagnosing, prognosing or monitoring the disease.

In some embodiments, this invention can provide methods for early detection of disease in a subject by liquid biopsy. The methods include obtaining a biological sample from the subject, isolating extracellular matrix bodies from the sample, and determining the presence of the disease in the subject from a level of the isolated extracellular matrix bodies or a level of a biomarker contained in the extracellular matrix bodies. The presence of the disease in the subject may be determined before any one of:
onset of clinical signs and symptoms of the disease in the subject,
treatment for the disease is recommended or administered based on clinical examination of the subject, and
disease is detected in the subject by needle or tissue biopsy.

In certain embodiments, this invention includes methods for treating the subject for the disease by any one or more of surgery, drug therapy, therapeutic radiation, and chemotherapy. A process for diagnosing, prognosing or monitoring a disease in a subject can include steps for separating, isolating or enriching extracellular matrix bodies in a biological fluid sample of the subject, determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid, comparing the level of the biomarkers to a reference level based on a control group of subjects, diagnosing, prognosing or monitoring the disease in the subject, and treating the subject for the disease by any one or more of surgery, drug therapy, therapeutic radiation, and chemotherapy.

### Preparing Samples for Liquid Biopsy

Aspects of this invention include isolating and preserving the composition and properties of extracellular matrix bodies from a biological fluid or material. By preserving the composition and properties of extracellular matrix bodies isolated or extracted from a biological sample, fluid or material, the extracellular matrix bodies can be used for diagnosis or medical information, or for monitoring biochemical or biological processes or changes of the sample material. Embodiments of this invention can be used to isolate, extract, and utilize extracellular matrix bodies that are a source of multiple and specific biomarkers.

A sample fluid of this disclosure may contain a carrier fluid, a biofluid, and/or reagents of interest. Examples of a carrier include water, purified water, saline solution, and organic solvents. A sample fluid may contain a gelling agent, a surfactant, or reagents for interacting with biological components.

Additional methods of this disclosure include preparing a biological sample for a diagnostic, prognostic, clinical or therapeutic use by isolating extracellular matrix bodies from the biological sample. The biological sample may be composed of bodily fluid, homogenized tissue, lysed cells, and/or lysed vesicles.

Examples of biological fluid include any bodily fluid, whole blood, blood plasma, blood serum, cerebrospinal fluid, urine, saliva, sweat, tears, synovial fluid, pleural fluid, gastric fluid, peritoneal fluid, breast milk, nipple aspirate, semen, amniotic fluid, vitreous, aqueous humor, lymph, bile, cerumen, chyle, chyme, endolymph, perilymph, exudates, feces, vaginal fluid, pericardial fluid, amniotic fluid, nasal fluid, otic fluid, ejaculate, gastric acid, gastric juice, mucus, pericardial fluid, pus, rheum, sebum, serous fluid, smegma, sputum, synovial fluid, vaginal secretion, menstrual effluent, vomit and combinations thereof.

Embodiments of this invention include methods for preparing a biological sample for a medical, diagnostic or prognostic use by isolating extracellular matrix bodies from the biological sample. Extracellular matrix bodies of biological sample such as a bodily fluid may be isolated by affinity methods as described herein.

A kit of this invention for a medical, diagnostic or prognostic use of extracellular matrix bodies may contain one or more reagents for measuring a biomarker level or quantity as disclosed herein, and comparing the biomarker level to a control. A kit of this invention may contain one or more reagents for measuring one or more proteins disclosed in Table 1 herein.

In some embodiments, this invention provides methods for preparing samples for obtaining medical information, or for diagnosis, prognosis or monitoring of disease by contacting a tissue sample with a buffer or reagent to release extracellular matrix components such as extracellular matrix bodies.

### Extracted Compositions and Methods of Use

A composition of this invention may be composed of separated, isolated or enriched extracellular matrix bodies, which may be used in treatment of the human or animal body. The extracellular matrix bodies may be associated with pathology of a disease.

A composition of extracellular matrix bodies, isolated and/or extracted, can be combined with a pharmaceutical carrier and one or more pharmaceutical excipients.

A composition of this invention may be composed of a fraction of a bodily fluid in which extracellular matrix bodies have been separated, isolated or enriched. The composition may be used in treatment of the human or animal body. The extracellular matrix bodies may be associated with pathology of a disease.

In further embodiments, a composition may comprise a sample from which extracellular matrix bodies have been removed by the isolation and/or extraction processes for use in the treatment of the human or animal body. In certain embodiments, at least 25%, or at least 50%, or at least 75%, or at least 90%, or substantially all of the extracellular matrix bodies of a sample have been removed by the isolation and/or extraction processes herein for use in the treatment of the human or animal body.

Numbered embodiments of this invention may include:

1) A process for separating, isolating or enriching extracellular matrix bodies in a biological fluid, the process comprising capturing the extracellular matrix bodies on a substrate.

2) The process of embodiment 1), wherein the substrate is a solid and comprises one or more immobilized capture moieties for binding and immobilizing the extracellular matrix bodies.

3) The process of any of embodiments 1-2, wherein the capturing comprises contacting the biological fluid with the substrate.

4) The process of any of embodiments 1-3, wherein the capturing comprises adding capture moieties to the biological fluid and contacting the biological fluid with the substrate.

5) The process of any of embodiments 1-4, wherein the biological fluid is incubated with the substrate, and wherein the substrate comprises capture moieties.

6) The process of any of embodiments 1-5, wherein the substrate is of any shape in the form of a bead, a gel, a magnetic bead, a paramagnetic bead, a plate, a well, a membrane, a particle, a sheet, or a fiber.

7) The process of any of embodiments 1-6, wherein the substrate is composed of an inorganic material, a polymeric material, an organic material, a metal, a glass, or a combination thereof.

8) The process of any of embodiments 1-7, wherein the biological fluid is processed before the capturing on the substrate to remove cells and cell debris.

9) The process of any of embodiments 1-8, comprising washing the substrate to remove the biological fluid and any non-bound components from the substrate.

10) The process of any of embodiments 1-9, comprising eluting the immobilized extracellular matrix bodies from the substrate, wherein the extracellular matrix bodies have a principal size from about 1 micrometer to 200 micrometers, or from about 4 micrometers to 200 micrometers.

11) The process of any of embodiments 1-10, comprising tagging the extracellular matrix bodies in the biological fluid for capturing on the substrate.

12) The process of embodiments 11, wherein the tagging uses epitope tags, affinity tags, fluorescent tags, or a combination thereof.

13) The process of any of embodiments 2-12, wherein the capture moieties bind to any of a protein, an extracellular matrix protein, a polypeptide, a lipid molecule, a lipoparticle, a carbohydrate, or a nucleic acid of the extracellular matrix bodies.

14) The process of any of embodiments 2-13, wherein the capture moieties have specific or non-specific interactions with a component of the extracellular matrix bodies.

15) The process of any of embodiments 2-14, wherein the capture moieties are antibodies, metal ions, or dyes.

16) The process of any of embodiments 1-15, wherein at least a majority of the extracellular matrix bodies are captured from the biological fluid.

17) The process of any of embodiments 1-16, wherein at least a majority of the extracellular matrix bodies are captured from the biological fluid with an absence of cells.

18) The process of any of embodiments 1-17, wherein substantially all of the extracellular matrix bodies are captured from the biological fluid.

19) The process of any of embodiments 1-18, wherein substantially all of the extracellular matrix bodies are captured from the biological fluid with an absence of cells.

20) The process of any of embodiments 1-19, wherein the captured extracellular matrix bodies are a biomarker or contain biomarkers for medical, diagnostic or prognostic information.

21) The process of any of embodiments 1-20, wherein at least a component of the extracellular matrix bodies is captured from the biological fluid.

22) The process of any of embodiments 1-21, wherein at least a protein component of the extracellular matrix bodies is captured from the biological fluid.

23) The process of any of embodiments 1-22, further comprising additional separating, isolating or enriching of the eluted extracellular matrix bodies by microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

24) The process of any of embodiments 1-23, wherein the extracellular matrix bodies are captured using capture moieties with affinity to one or more protein components of the biological fluid selected from Table 1.

25) The process of any of embodiments 1-24, further comprising adding a reagent to the biological fluid, wherein the reagent is for precipitating the extracellular matrix bodies.

26) The process of any of embodiments 1-25, wherein the extracellular matrix bodies when eluted and re-suspended are at least 5-fold, or at least 10-fold, or at least 100-fold enriched in concentration as compared to the biological fluid.

27) The process of any of embodiments 1-26, wherein the extracellular matrix bodies are associated with a pathology or disease.

28) The process of any of embodiments 1-27, wherein the biological fluid is any one of whole blood, blood plasma, blood serum, cerebrospinal fluid, vitreous, aqueous humor, breast milk, nipple aspirate, urine, saliva, sweat, tears, synovial fluid, pleural fluid, gastric fluid, peritoneal fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof.

29) The process of any of embodiments 1-28, further comprising determining a level of a biomarker of the separated, isolated or enriched extracellular matrix bodies.

30) The process of embodiments 29, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid.

31) The process of embodiments 30, wherein the level of the substance is determined by any of microscopy, immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid sequencing, next generation sequencing, multi-well automated versions thereof, and combinations thereof.

32) A composition comprising extracellular matrix bodies captured by the process of any one of embodiments 1-31.

33) The composition of embodiments 32, wherein the extracellular matrix bodies are complexed with a tag or a solid substrate.

34) The composition of any of embodiments 32-33, wherein the extracellular matrix bodies are associated with pathology of a disease.

35) The composition of any of embodiments 32-34, for use in therapy of a human or animal body.

36) A method for preparing a biological sample for a medical, diagnostic or prognostic use, the method comprising isolating extracellular matrix bodies from the biological sample, wherein the extracellular matrix bodies have a principal size from about 1 micrometer to 200 micrometers, or from about 4 micrometers to 200 micrometers.

37) The method of embodiment 36, wherein the biological sample is composed of a bodily fluid.

38) The method of embodiments 37, wherein the bodily fluid is any of whole blood, blood plasma, blood serum, cerebrospinal fluid, vitreous, aqueous humor, breast milk, nipple aspirate, urine, saliva, sweat, tears, synovial fluid, pleural fluid, gastric fluid, peritoneal fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof.

39) The method of any of embodiments 36-38, wherein the extracellular matrix bodies are isolated by any of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

40) The method of any of embodiments 36-39, comprising capturing the extracellular matrix bodies on a solid substrate.

41) The method of any of embodiments 36-40, wherein the capturing comprises adding capture moieties to the biological sample and contacting the biological sample with the substrate.

42) A process for diagnosing, prognosing or monitoring a disease in a subject, the process comprising
separating, isolating or enriching extracellular matrix bodies in a biological fluid sample of the subject;
determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid; and
comparing the level of the biomarkers to a reference level based on a control group of subjects, and diagnosing, prognosing or monitoring the disease in the subject. The separating, isolating or enriching may be performed according to the steps of any of embodiments 1-41.

43) The process of embodiments 42, wherein the separated, isolated or enriched extracellular matrix bodies comprise biomarkers in the form of proteins, extracellular matrix proteins, polypeptides, lipids, lipoparticles, carbohydrates, nucleic acid molecules, DNA, or an expression level of a nucleic acid.

44) The process of any of embodiments 42-43, wherein the separating, isolating or enriching extracellular matrix bodies in a biological fluid sample of the subject comprises performing a process according to any one of claims 1 to 31.

45) The process of any of embodiments 42-44, comprising treating the subject for the disease by any one or more of surgery, drug therapy, therapeutic radiation, and chemotherapy.

46) A kit for separating, isolating or enriching extracellular matrix bodies in a biological fluid, comprising:
a container for holding the biological fluid; and
one or more reagents for capturing the extracellular matrix bodies on a solid substrate.

47) The kit of embodiment 46, wherein the reagents have affinity for a protein component of the extracellular matrix bodies.

48) The kit of embodiment 46 or embodiment 47, wherein the reagents are suitable for any of microscopy, immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid sequencing, next generation sequencing, multi-well automated versions thereof, and combinations thereof.

All publications including patents, patent application publications, and non-patent publications referred to in this description are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The terms “a,” “an,” “the,” and similar terms describing the invention, and in the claims, are to be construed to include both the singular and the plural.

### EXAMPLES

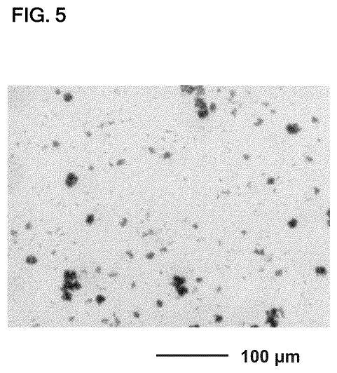

Example 1. Extracellular matrix bodies isolated from human plasma. Extracellular matrix bodies were isolated from human blood plasma. FIG. 5 is a representative fluorescence photomicrograph of extracellular matrix bodies isolated from human blood plasma and shows that the extracellular matrix bodies were morphologically and physiologically distinct from other components of the plasma. The extracellular matrix bodies in FIG. 5 have various shapes with principal sizes of up to about 30 micrometers or more.

The extracellular matrix bodies of FIG. 5 were isolated from human blood plasma by immunoprecipitation with a galectin-3-binding protein antibody conjugated to a magnetic bead (10.1 ng/ml of Gal3BP). A control antibody (IgG) showed no isolation of extracellular matrix bodies (not shown). Eluates from the beads were collected in elution buffer for this image.

Extracellular matrix bodies isolated by immunoaffinity assays were imaged on glass slides with immunofluorescence of extracellular matrix proteins. Briefly, two equal samples of the isolated extracellular matrix bodies were separately mixed with equal volumes of EDC and placed on separate glass slides. The slides were incubated for 30 minutes on ice and then an overnight at 37° C. in an incubator. The following day the slides were incubated for 1 h either with human Galectin-3BP antibody (Human Galectin-3BP/AF2226 from R&D systems) for Galectin-3-binding protein or IgG (Sigma) at 1/40 dilution. The slides were then washed with 0.1% Tween 20 in 1×PBS once and incubated with secondary donkey anti-goat Dylight 680 from Invitrogen at 1/200 dilution in 1×PBS for an hour. The slides were washed twice with 0.1% Tween20/1×PBS. The samples were processed for microscopy with mounting media and glass coverslips. The photomicrographs were captured by using wide-field fluorescence microscopy.

Example 2. Extracellular matrix bodies isolated from human (CSF). Extracellular matrix bodies were isolated from human cerebrospinal fluid.

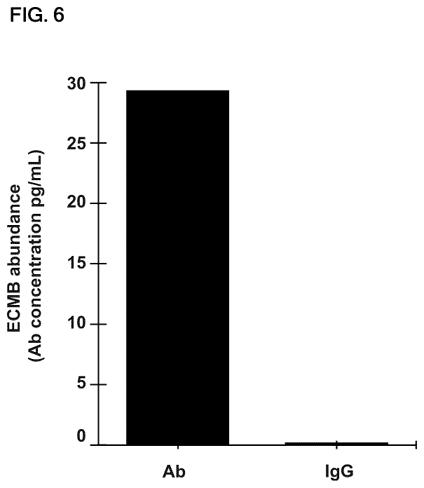

FIG. 6 shows a graph of the quantities of extracellular matrix bodies isolated from human cerebrospinal fluid by immunoprecipitation with probe antibodies (Ab) conjugated to magnetic beads. The relative abundance of extracellular matrix bodies (y-axis) was proportional to the probe antibody concentration (pg/ml). The enzyme linked immunosorbent assay (ELISA) detected and quantified a protein in the extracellular matrix bodies. A negative control antibody (IgG) showed no isolation of extracellular matrix bodies.

The graph in FIG. 6 shows the relative abundance of extracellular matrix bodies (y-axis) determined by analyzing representative fluorescence proportional to the amount of specific protein present. The relative abundance of extracellular matrix bodies (y-axis) was proportional to the probe antibody (Ab) concentration (pg/ml). A negative control antibody (IgG) showed no isolation of extracellular matrix bodies. These data show that immunoprecipitation of extracellular matrix bodies was far greater than for the negative control. The probe antibody Ab was different from Gal3BP and fibronectin antibodies.

Example 3. Extracellular matrix bodies isolated from human blood plasma. Extracellular matrix bodies were isolated from human blood plasma.

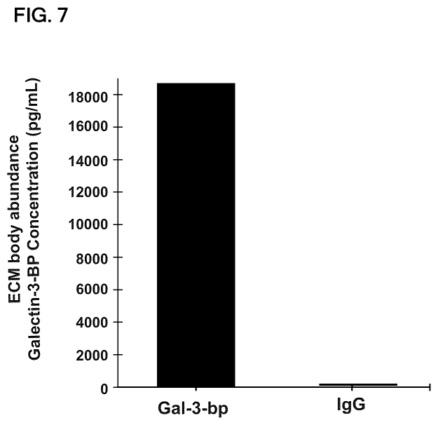

FIG. 7 shows a graph of the quantities of extracellular matrix bodies isolated from human blood plasma by immunoprecipitation with a galectin-3-binding protein antibodies conjugated to magnetic beads (18.7 ng/ml of Gal3BP). The relative abundance of extracellular matrix bodies (y-axis) was proportional to the Gal3BP antibody concentration (pg/ml). A negative control antibody (IgG) showed no isolation of extracellular matrix bodies. These data show that immunoprecipitation of extracellular matrix bodies was far greater than for the negative control.

Example 4. Extracellular matrix bodies isolated from human blood plasma exhibited a range of principal sizes. Extracellular matrix bodies were isolated from human blood plasma and exhibited a range of principal sizes.

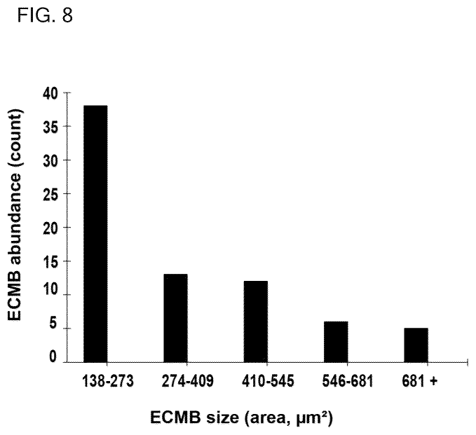

FIG. 8 shows a histogram of the relative abundances and sizes of extracellular matrix bodies isolated from human blood plasma by immunoprecipitation with a galectin-3-binding protein antibody conjugated to a magnetic bead (18.7 ng/ml of Gal3BP). The sizes of extracellular matrix bodies in FIG. 8 ranged from 138 (μm)² (about 13.2 μm diameter) to 681 (μm)² (about 29 μm diameter) and greater. Smaller particles were also observed with principal sizes in the range from about 2 to 13 μm.

Example 5. Extracellular matrix bodies isolated from vitreous humor. Fibronectin protein (gene FN1) found in extracellular matrix bodies was targeted with antibody-linked magnetic beads. Bovine vitreous humor in buffer was incubated with magnetic beads conjugated with the fibronectin antibody. For control, IgG antibody was conjugated to separate magnetic beads. IgG was not present or accessible in the extracellular matrix bodies, and served as negative control. The relative amounts of fibronectin and control IgG isolated were determined by ELISA. The quantity of extracellular matrix bodies isolated was directly dependent on the amount of fibronectin detected.

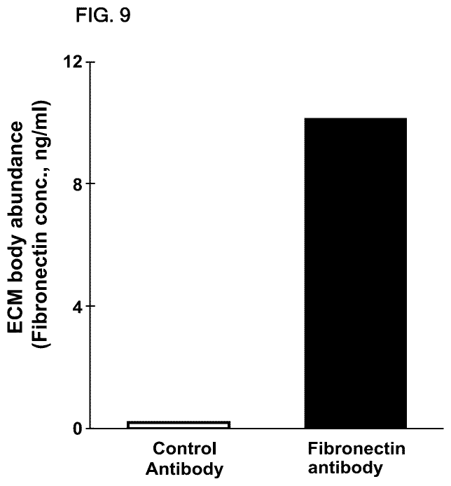

FIG. 9 shows a graph of the quantities of extracellular matrix bodies that were isolated from a biological fluid sample, bovine vitreous humor, using immunoprecipitation. FIG. 9 shows the relative abundance of fibronectin protein by concentration (ng/ml, y-axis) after the incubation. Also shown is the fibronectin protein concentration detected using a control IgG antibody, which is essentially zero. These data show that fibronectin immunoprecipitation of extracellular matrix bodies (10.1 ng/ml, black bar) was far greater than for negative control IgG antibody (0.2 ng/ml, white bar).

Thus, this example demonstrates a method for isolating extracellular matrix bodies from a biological fluid via immunoaffinity capture.

Example 6. Relative abundance of extracellular matrix bodies isolated by immunoprecipitation was determined by microscopy. This example demonstrates a method for determining by microscopy the relative abundance of extracellular matrix bodies isolated by immunoprecipitation from a biological fluid.

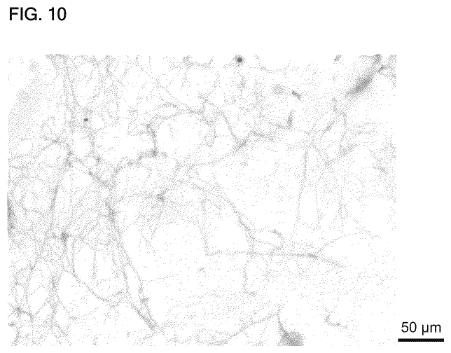

FIG. 10 shows a representative fluorescence photomicrograph of native bovine vitreous humor. Extracellular matrix bodies were present and visualized with immunofluorescent staining for fibronectin (black, anti-fibronectin antibody, Alexa 488). The bovine vitreous humor was fixed to a glass slide, incubated with a fibronectin antibody conjugated to a fluorophore (Alexa 488), and imaged with wide-field fluorescence microscopy (FITC).

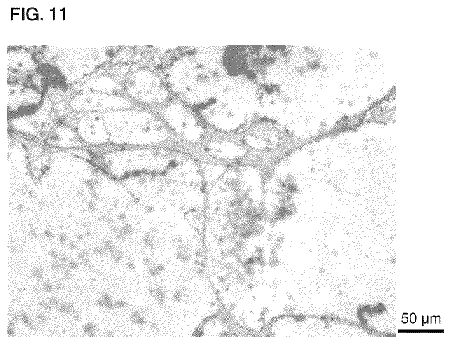

FIG. 11 shows a representative fluorescence photomicrograph of extracellular matrix bodies extracted from a biological fluid, bovine vitreous humor, after immunoprecipitation with fibronectin antibody. Antibodies specific to fibronectin and conjugated to magnetic beads were incubated with bovine vitreous humor to extract extracellular matrix bodies. The sample was washed for non-specific binding and visualized on a glass slide for imaging.

To determine the amount of extracellular matrix bodies in the isolate, the extracellular matrix bodies were labeled with antibodies for fibronectin and measured by immunohistochemistry and immunofluorescence imaging. FIG. 11 shows signal for fibronectin in black stain (Alexa 488, FITC). This photomicrograph shows that extracellular matrix bodies were isolated by immunocapture. The black stain signal in the image shows biological material belonging to extracellular matrix bodies. The relative abundance of extracellular matrix bodies can be determined by image analysis.

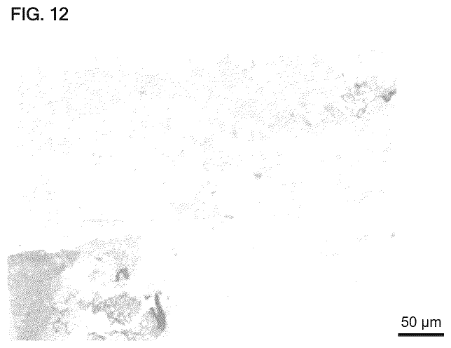

FIG. 12 shows a representative fluorescence photomicrograph for corresponding negative control as compared to FIG. 11. FIG. 12 confirms that essentially no extracellular matrix bodies were found after immunoprecipitating with a control IgG antibody. Thus, the extracellular matrix bodies isolated in FIG. 11 were specifically enriched relative to the native fluid.

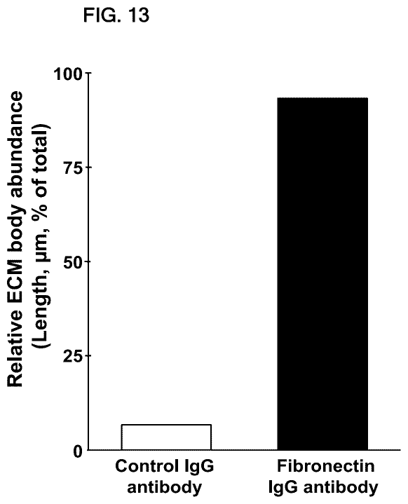

FIG. 13 shows a graphical representation of the relative amounts of extracellular matrix bodies isolated from bovine vitreous humor using immunoprecipitation with a fibronectin antibody conjugated to a magnetic bead. The graph shows the relative abundance of extracellular matrix bodies (y-axis) determined by analyzing multiple (n=3) representative fluorescence photomicrographs using computational software (ImageJ). Analysis was done with representative photographic fields for each 20× photomicrographic image. The relative amount of extracellular matrix bodies was measured as a percent of total.

FIG. 13 shows that the fibronectin immunoprecipitation sample contained 93.4% of the total of extracellular matrix bodies (black bar).

In an additional experiment, the fibronectin immunoprecipitation sample contained 93.1% of the total of extracellular matrix bodies (not shown).

Thus, immunoaffinity capture is a useful method for isolating extracellular matrix bodies from a biological fluid. Extracellular matrix bodies can be separated, isolated, and/or enriched from a bodily fluid by immunoprecipitation.

Example 7. Tissue preparation and processing for bovine vitreous humor bodily fluid. Samples of bovine vitreous humor bodily fluid were prepared. Bovine eyes for dissection were placed in a 100 mm plastic petri dish on ice to prevent nucleic acid and protein degradation. Using an SZX-16 stereo dissecting microscope (Olympus), orbital fat and extraocular muscles attached to the globe were removed. The globe was rinsed with 5 ml ice-cold Tris Buffered Saline (TBS) containing 50 mM Tris-HCl, 150 mM NaCl (pH 8.0) for 1 minute at 4° C. Vitreous was dissected by making a sclerotomy incision 4 mm posterior to the limbus using a 16 g needle and then making a circumferential sagittal incision with scissors to separate the globe into an anterior and posterior cup. Scissors were used to cut and remove the formed vitreous and to sever adhesions between vitreous and ocular structures. Vitreous contamination by uveal tissue or neural retina was avoided. Vitreous samples were rinsed with TBS (pH 8.0) for 1 min at 4° C. Vitreous specimens collected were placed in 1.5 ml centrifuge tubes frozen at −80° C. until use.

Example 8. Extracellular matrix bodies isolated from vitreous humor by immunoaffinity capture. This example shows a method for isolating extracellular matrix bodies from a biological fluid via immunoaffinity capture.

Extracellular matrix bodies, which contain fibronectin, were isolated from a bodily fluid by immunoprecipitation using fibronectin antibodies with a magnetic bead kit (Crosslink Magnetic IP, Thermofisher, #88805). The amount of fibronectin protein measured is directly related to the amount of the extracellular matrix bodies. Antibodies were conjugated to the magnetic beads following manufacturer's instructions. 100 μl of each antibody solution (Novus, NBP1-91258 rabbit antibody, 1 mg/ml for fibronectin) and rabbit IgG for negative control (Sigma, R1933-5 ml, rabbit serum) were prepared by diluting each antibody in 1× Modified Coupling Buffer and 1× Wash buffer to a final concentration of 5 μg.

For each immunoprecipitation, 25 μl of magnetic beads was washed twice with 500 μl of 1× Modified Coupling buffer. The diluted antibody was added to the beads and gently vortexed and incubated for 15 minutes at room temperature on an Eppendorf thermomixer. The beads were collected by a magnetic stand (BioRad, Sure Beads Magnetic Rack). The supernatant was removed by pipetting and discarded. The beads were further mixed with 100 μl of 1× Modified Coupling buffer and gently vortexed and collected on a magnetic stand. The supernatant was removed, and the step was repeated thrice.

Crosslinker Disuccinimidyl suberate (DDS) was diluted with Dimethyl Sulfoxide (DMSO) at 1:100 to make 0.25 mM DSS. The following components were added to the beads to make the DSS at 10× molar excess: 2.5 μl of 20× Modified Coupling buffer, 4 μl of 0.25 mM DSS and 43.5 μl of ultrapure water. The crosslinking reaction was incubated for 30 minutes at room temperature on a mixer. The beads were collected on the magnetic stand and the supernatant (flow through) was saved for confirming the antibody crosslinking. For removing the non-crosslinked antibody and quench the crosslinking reaction, 100 μl elution buffer was added to the beads and incubated for 5 minutes at room temperature. The antibody crosslinked magnetic beads were washed twice at room temperature by suspending the beads in 300 μl of Wash buffer and collecting the beads on the magnetic stand, the wash buffer was discarded.

The fibronectin antibody and IgG control crosslinked magnetic beads were mixed with 500 μl of homogenized bovine vitreous humor and incubated for 1 h at room temperature. The beads were collected by a magnetic stand and washed twice with Wash buffer as described above. After each mixing the beads and washing with buffer, the tubes containing the beads were put on the magnetic stand until the supernatants were clear. The beads were mixed with 500 μl ultrapure water and collected on a magnetic stand. The wash water was discarded. Finally, to recover the Fibronectin that was bound to the antibody crosslinked magnetic beads, 100 μl Elution buffer was added to the beads, incubated 5 minutes at room temperature on a mixer. The IgG control crosslinked magnetic beads were also eluted with 100 μl of Elution buffer. The eluates were collected from the beads by placing the tubes on the magnetic stand. The samples were stored at 4° C. and immediately analyzed or frozen at −20° C.

Example 9. Fixing and staining extracellular matrix bodies for detection by microscopy. Glycosaminoglycan composition of extracellular matrix bodies from bovine vitreous humor (BVH) was detected by staining. Bovine vitreous humor was fixed to a poly-L-lysine coated superfrost plus glass slide using EDC-crosslinking. To prepare for sample staining, 1% Alcian Blue in 3% acetic acid, which detects GAGs such as hyaluronic acid, was applied to the demarked region and incubated in a dark chamber for 30 minutes and then thoroughly rinsed with distilled water at room temperature. After the incubation, the solution was removed with a pipette or decanted. Samples were gently washed 2-3 times with acidified (3% glacial acetic acid) diluted in deionized water (DI) by pipetting the DI water onto a corner of the demarked square and then removing the DI water in the same fashion at room temperature. The demarked square was coved with a 40% glycerol solution and an adequate amount of mounting medium and covered with a cover slip. The cover slip edges were sealed with nail polish and then dried for 15 minutes at room temperature prior to imaging. A Zeiss Axiovert 200 wide-field microscope with Zen imaging software was used to capture images within 1-2 hours of Alcian blue/PSR staining on the glass slide.

Example 10. Quantitation of extracellular matrix bodies after immunoaffinity immunoprecipitation using ELISA for fibronectin. An ELISA kit (ABCAM) was used following the manufacturer's instructions to quantitate the amount of fibronectin. Standards were serially diluted from a stock of purified fibronectin standard protein (32 ng/ml) provided in the kit. The samples (50 μl of purified fibronectin and 50 μl of IgG control) were separately added to each well. On one column of a 96 well plate, 50 μl of each diluted standard was added to each well of the plate (A1 to H1 of a plate coated with Fibronectin antibody provided in the kit). Equal volume of 50 μl of antibody cocktail was added to each well. The plate was sealed and incubated for 1 h at room temperature on a plate shaker. After incubation, the wells were washed three times with the wash buffer and after the last wash the wash buffer was completely removed. The plate was gently tapped on a paper towel to remove any excess of liquid. One hundred microliters (100 μl) of TMB development solution were added to each well and incubated for 10 minutes in the dark. After the color development was completed 100 μl of stop solution was added to each well. The plate was read on a plate reader at 450 nm. The absorbance of the fibronectin standard proteins were recorded and a calibration curve was generated. The amount of fibronectin reflecting the quantity of extracellular matrix bodies and the control were determined by plotting the absorbance readings on the standard curve. Because the amount of fibronectin protein was directly dependent on the amount of extracellular matrix bodies, the data was represented in a graph by plotting the amount of fibronectin protein.

Example 11. Quantitation of extracellular matrix bodies by immunofluorescence after enrichment by immunoaffinity assays. Extracellular matrix bodies isolated by immunoaffinity assays were visualized by labelling extracellular matrix proteins and imaging the specimens with microscopy on glass slides. Equal volumes and concentrations (20 μl) of purified extracellular matrix bodies (Fibronectin concentration, 3.6 pg/ml) obtained by fibronectin immunoprecipitation and IgG control were each mixed with equal volumes of EDC for fixation and placed on the glass slides. The slides were incubated for 30 minutes on ice and then an overnight at 37° C. in an incubator. The following day the slides were incubated for 1 h with either rabbit antibody for Fibronectin (Novus) or isotype matched IgG (Sigma) at 1/200 dilution. The slides were then washed with 0.1% Tween 20 in 1×PBS multiple times and further were incubated with secondary goat anti-rabbit Alexa Fluor 488 IgG (H+L) (Invitrogen) at 1/700 dilution in 1×PBS for an hour. The slides were washed twice with 0.1% Tween20/1×PBS and the images were taken by using wide-field fluorescence microscopy.

Color bright field images were captured on a Ziess inverted phase contrast Axiovert 200 microscope equipped with an axiocam 105 color camera and images were processed with Zen software (Zeiss, version 4.3).

1-48. (canceled)

49. A process for separating, isolating or enriching extracellular matrix bodies in a biological fluid, the process comprising capturing the extracellular matrix bodies on a substrate, optionally wherein:
a) the substrate is:
i) a solid and comprises one or more immobilized capture moieties for binding and immobilizing the extracellular matrix bodies;
ii) of any shape in the form of a bead, a gel, a magnetic bead, a paramagnetic bead, a plate, a well, a membrane, a particle, a sheet, or a fiber; and/or
iii) composed of an inorganic material, a polymeric material, an organic material, a metal, a glass, or a combination thereof;
b) the capturing comprises:
i) contacting the biological fluid with the substrate; and/or
ii) adding capture moieties to the biological fluid and contacting the biological fluid with the substrate;
c) the biological fluid is:
i) incubated with the substrate, and wherein the substrate comprises capture moieties; and/or
ii) the biological fluid is processed before the capturing on the substrate to remove cells and cell debris; and/or
d) the process further comprises:
i) washing the substrate to remove the biological fluid and any non-bound components from the substrate;
ii) eluting the immobilized extracellular matrix bodies from the substrate, wherein the extracellular matrix bodies have a principal size from about 1 micrometer to 200 micrometers, or from about 4 micrometers to 200 micrometers; and/or
iii) tagging the extracellular matrix bodies in the biological fluid for capturing on the substrate, optionally wherein the tagging uses epitope tags, affinity tags, fluorescent tags, or a combination thereof.

50. The process of claim 49, wherein the capture moieties bind to any of a protein, an extracellular matrix protein, a polypeptide, a lipid molecule, a lipoparticle, a carbohydrate, or a nucleic acid of the extracellular matrix bodies, optionally wherein the capture moieties are antibodies, metal ions, or dyes.

51. The process of claim 49, wherein at least a majority of or substantially all of the extracellular matrix bodies are captured from the biological fluid, optionally with an absence of cells.

52. The process of claim 49, wherein the captured extracellular matrix bodies are a biomarker or contain biomarkers for medical, diagnostic or prognostic information.

53. The process of claim 49, further comprising additional separating, isolating or enriching of the eluted extracellular matrix bodies by microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

54. The process of claim 49, wherein the extracellular matrix bodies are captured using capture moieties with affinity to one or more biomarkers selected from the group consisting of: IGHG4, PIGR, IGHA1, IGLV4-60, IGHD, HBB, HPR, IGKV1-6, IGHM, HBA1, CFB, LPA, F12, B4E1Z4, IGHV3-33, SERPINF2, IGHV2-70D, JCHAIN, IGHV6-1, TLN1, FGA, THBS1, IGHA2, CFH, IGHV5-51, IGHV4-39, IGKV3-20, IGHV3-53, ITIH1, A2M, CFD, CRP, CD5L, LGALS3BP, IGKV3-15, PON1, ORM1, SLC4A1, APOC4-APOC2, IGKV2-29, FGB, IGLC2, IGHV3OR15-7, IGHA2, IGLV8-61, IGHV3-21, IGLV4-69, IGLV1-44, C4B, C7, ACTB, IGLV3-21, PGLYRP2, IGHV1-18, IGKV2D-28, IGKV2-24, FBLL1, IGKC, IGHV3-9, ITIH4, AHSG, AGT, IGHV1-69, IGHV1-3, SPTA1, IGLV2-11, IGKV1D-33, LRG1, IGHV3-72, FGG, APOC1, LYZ, IGKV1-5, IGKV1-27, IGHV1-46, C1R, IGKV1-16, C4BPB, IGFALS, IGKV4-1, PF4, IGHV1OR15-1, IGKV1D-12, IGKV2-30, IGHV3-74, SERPING1, IGHV3-43D, APOH, C1S, PLG, IGLV3-9, IGHV3-49, APOL1, SERPINC1, CFP, AMBP, C6, IGHV3-38, IGKV2D-29, IGHV4-28, ADIPOQ, IGHV3-73, IGHG3, CPN1, PROS1, IGHG2, IGHV7-4-1, F2, IGLV3-10, IGLV6-57, CFH, ALB, HRG, FN1, C3, C1QB, F5, GC, IGLV1-51, PLTP, EFEMP1, SERPINA5, CPN2, IGKV1-13, IGKV3-11, IGLV3-25, SHBG, FCGBP, AZGP1, ING4, APOD, HPX, CD14, APOE, IGLV3-19, C9, APOM, IGKV3-7, IGLL5, ITIH2, IGKV1-9, ECM1, IGHV3OR16-12, CLEC3B, APOB, CFHR2, IG-unk, IGKV1D-39, TTR, FBLN1, IGHV3-64, KLKB1, LUM, VTN, C5, PZP, SERPIND1, F13B, HABP2, APOA2, SERPINA6, IGLL1, APOA4, IGHV3-66, IGKV6D-21, IGHV3-13, ALB, IGKV1D-37, IGKV1-17, ITIH3, APCS, CPB2, CFI, C1QC, SERPINF1, TGFB1, C8G, IGHV2-5, APOA1, F9, CLU, KNG1, IGLV1-40, SERPINA1, C8A, IGHG1, SELENOP, HP, IGHV4-38-2, SERPINA4, LBP, ORM2, FN1, F10, APOC3, CFHR1, ATRN, B2M, VWF, F13A1, AFM, GPX3, FCN3, RBP4, C8B, SERPINA3, PRG4, C4A, SAA2-SAA4, SERPINA7, CP, A1BG, C2, IGHV1-2, HGFAC, FETUB, GSN, SERPINA1, C1QA, C4BPA, IGKV3OR2-268, HSPG2, FGG, FGA, FGB, APOC1, LYZ, ANG, IGFBP5, C1QB, F5, H1-4, HBA1, KRT10, H1-0, QSOX1, HABP2, KRT9, KRT2, VTN, H4C1, KRT1, C8B, C1QA, MTHFD2, CFHR1, ACTB, HRG, EEF1A1, H2AC4, MMP2, TIMP2, ATP5F1B, HGFAC, ENO1, HSPG2, COL18A1, APOE, CFH, RARRES2, SERPINA5, SAA4, PEBP4, C1QC, CHI3L1, PON1, CLU, SERPIND1, PCOLCE, C8A, APOM, APOA1, APOD, ECM1, SELENOP, SERPINA4, KNG1, C9, PROS1, CFHR2, SPON1, CLSTN1, APP, GPX3, RNASE1, IGFBP6, AL645922.1, ITIH2, C5, GSN, APOA2, RBP3, HP, C3, APOA4, C6, APLP2, TTR, and PLG.

55. The process of claim 49, further comprising adding a reagent to the biological fluid, wherein the reagent is for precipitating the extracellular matrix bodies.

56. The process of claim 49, wherein the extracellular matrix bodies when eluted and re-suspended are at least 5-fold, or at least 10-fold, or at least 100-fold enriched in concentration as compared to the biological fluid.

57. The process of claim 49, wherein the extracellular matrix bodies are associated with a pathology or disease.

58. The process of claim 49, wherein the biological fluid is any one of whole blood, blood plasma, blood serum, cerebrospinal fluid, vitreous, aqueous humor, breast milk, nipple aspirate, urine, saliva, sweat, tears, synovial fluid, pleural fluid, gastric fluid, peritoneal fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof.

59. The process of claim 49, further comprising determining a level of a biomarker of the separated, isolated or enriched extracellular matrix bodies.

60. The process of claim 59, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid.

61. The process of claim 60, wherein the level of the substance is determined by any of microscopy, immunostaining, fluorescence assay, chelate complexation, quantitative HPLC, spectrophotometry, antibody array, Western blot, immunoassay, immunoprecipitation, ELISA, LC-MS, LC-MRM, radioimmunoassay, mass spectrometry, 2D gel mass spectrometry, LC-MS/MS, RT-PCR, nucleic acid sequencing, next generation sequencing, multi-well automated versions thereof, and combinations thereof.

62. A method for preparing a biological sample by distinguishing extracellular matrix bodies for a medical, diagnostic or prognostic use, the method comprising isolating extracellular matrix bodies from the biological sample, wherein the isolation involves capturing the extracellular matrix bodies on a substrate, and wherein the extracellular matrix bodies have a principal size from about 1 micrometer to 200 micrometers, or from about 4 micrometers to 200 micrometers.

63. The method of claim 62, wherein the biological sample is composed of a bodily fluid, optionally wherein the bodily fluid is any of whole blood, blood plasma, blood serum, cerebrospinal fluid, vitreous, aqueous humor, breast milk, nipple aspirate, urine, saliva, sweat, tears, synovial fluid, pleural fluid, gastric fluid, peritoneal fluid, semen, amniotic fluid, lymph, bile, cerumen, chyle, chyme, endolymph, perilymph, exudates, feces, ejaculate, gastric acid, gastric juice, mucus, pericardial fluid, pus, rheum, sebum, serous fluid, smegma, sputum, synovial fluid, vaginal secretion, menstrual effluent, vomit, and combinations thereof.

64. The method of claim 62, wherein the extracellular matrix bodies are isolated by any of microfluidic separation, affinity chromatography, centrifugation, differential centrifugation, density gradient centrifugation, mesh filtration, diafiltration, tangential flow filtration, membrane filtration, immuno-affinity capture, magnetic bead capture, size exclusion chromatography, electrophoresis, AC electrokinetics, and combinations thereof.

65. The method of claim 62, wherein the capturing comprises adding capture moieties to the biological sample and contacting the biological sample with the substrate.

66. A process for diagnosing, prognosing or monitoring a disease in a subject, the process comprising:
a) separating, isolating or enriching extracellular matrix bodies in a biological fluid sample of the subject, wherein the separation, isolation or enrichment involves capturing the extracellular matrix bodies on a substrate;
b) determining a level of one or more biomarkers based on the separated, isolated or enriched extracellular matrix bodies, wherein the biomarker is the level of the extracellular matrix bodies, or the level of a substance found in the extracellular matrix bodies, wherein the substance is a protein, a polypeptide, a lipid molecule, a lipoparticle, a carbohydrate, a nucleic acid molecule, or an expression level of a nucleic acid; and
c) comparing the level of the biomarkers to a reference level based on a control group of subjects, and diagnosing, prognosing or monitoring the disease in the subject.

67. The process of claim 66, wherein the separated, isolated or enriched extracellular matrix bodies comprise biomarkers in the form of proteins, extracellular matrix proteins, polypeptides, lipids, lipoparticles, carbohydrates, nucleic acid molecules, DNA, or an expression level of a nucleic acid.

68. The process of claim 66, comprising treating the subject for the disease by any one or more of surgery, drug therapy, therapeutic radiation, and chemotherapy.
